SEQUENCE LISTING (72 sequences)

SEQ ID NO: 1 - protein, 18 aa, synthetic construct
  REGION 1..18: note = source = /note="Description of Artificial Sequence: Syntheticpeptide"
LPGCKWDLLIKQWVCDPL

SEQ ID NO: 2 - protein, 18 aa, synthetic construct
  REGION 1..18: note = source = /note="Description of Artificial Sequence: Syntheticpeptide"
FHDCKWDLLTKQWVCHGL

SEQ ID NO: 3 - protein, 12 aa, synthetic construct
  VARIANT 3: note = /replace="Tyr" or "Phe"
  MOD_RES 5: note = Any amino acid
  VARIANT 7: note = /replace="Ile"
  VARIANT 8: note = /replace="Arg" or "His"
  VARIANT 10: note = /replace="Arg" or "His" or "Ala" or "Val" or "Leu" or"Ile" or "Pro" or "Phe" or "Trp" or "Met"
  REGION 1..12: note = misc_feature - /note="Variant residues given in the sequence have nopreference with respect to those in the annotations forvariant positions"
  REGION 1..12: note = source = /note="Description of Artificial Sequence: Syntheticpeptide"
CKWDXLTKQKVC

SEQ ID NO: 4 - protein, 4 aa, synthetic construct
  REGION 1..4: note = source = /note="Description of Artificial Sequence: Syntheticpeptide"
GGGG

SEQ ID NO: 5 - protein, 23 aa, synthetic construct
  VARIANT 12: note = /replace="Val"
  REGION 1..23: note = misc_feature - /note="Variant residues given in the sequence have nopreference with respect to those in the annotations forvariant positions"
  REGION 1..23: note = source = /note="Description of Artificial Sequence: Syntheticpeptide"
GSGSATGGSGSSASSGSGSATHL

SEQ ID NO: 6 - protein, 23 aa, synthetic construct
  REGION 1..23: note = source = /note="Description of Artificial Sequence: Syntheticpeptide"
GSGSATGGSGSVASSGSGSATHL

SEQ ID NO: 7 - protein, 23 aa, synthetic construct
  REGION 1..23: note = source = /note="Description of Artificial Sequence: Syntheticpeptide"
GSGSATGGSGSSASSGSGSATHL

SEQ ID NO: 8 - protein, 11 aa, synthetic construct
  REGION 1..11: note = source = /note="Description of Artificial Sequence: Syntheticpeptide"
RASQGISNWLA

SEQ ID NO: 9 - protein, 7 aa, synthetic construct
  REGION 1..7: note = source = /note="Description of Artificial Sequence: Syntheticpeptide"
AASSLQS

SEQ ID NO: 10 - protein, 9 aa, synthetic construct
  REGION 1..9: note = source = /note="Description of Artificial Sequence: Syntheticpeptide"
QQYDSYPRT

SEQ ID NO: 11 - protein, 5 aa, synthetic construct
  REGION 1..5: note = source = /note="Description of Artificial Sequence: Syntheticpeptide"
SYWMS

SEQ ID NO: 12 - protein, 17 aa, synthetic construct
  REGION 1..17: note = source = /note="Description of Artificial Sequence: Syntheticpeptide"
YIKQDGNEKYYVDSVKG

SEQ ID NO: 13 - protein, 12 aa, synthetic construct
  REGION 1..12: note = source = /note="Description of Artificial Sequence: Syntheticpeptide"
EGILWFGDLPTF

SEQ ID NO: 14 - protein, 108 aa, synthetic construct
  REGION 1..108: note = source = /note="Description of Artificial Sequence: Syntheticpolypeptide"
DIQMTQSPSSLSASVGDRVTITCRASQGISNWLAWYQQKPEKAPKSLIYAASSLQSGVPSRFSGSGSGTDFTLTISSLQPEDFATYYCQQYDSYPRTFGQGTKVEIKR

SEQ ID NO: 15 - protein, 121 aa, synthetic construct
  REGION 1..121: note = source = /note="Description of Artificial Sequence: Syntheticpolypeptide"
EVQLVESGGGLVQPGGSLRLSCAASGFTFSSYWMSWVRQAPGKGLEWVAYIKQDGNEKYYVDSVKGRFTISRDNAKNSLYLQMNSLRAEDTAVYYCAREGILWFGDLPTFWGQGTLVTVSS

SEQ ID NO: 16 - protein, 509 aa, synthetic construct
  REGION 1..509: note = source = /note="Description of Artificial Sequence: Syntheticpolypeptide"
EVQLVESGGGLVQPGGSLRLSCAASGFTFSSYWMSWVRQAPGKGLEWVAYIKQDGNEKYYVDSVKGRFTISRDNAKNSLYLQMNSLRAEDTAVYYCAREGILWFGDLPTFWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGLPGCKWDLLIKQWVCDPLGSGSATGGSGSGASSGSGSATGSLPGCKWDLLIKQWVCDPL

SEQ ID NO: 17 - protein, 511 aa, synthetic construct
  REGION 1..511: note = source = /note="Description of Artificial Sequence: Syntheticpolypeptide"
EVQLVESGGGLVQPGGSLRLSCAASGFTFSSYWMSWVRQAPGKGLEWVAYIKQDGNEKYYVDSVKGRFTISRDNAKNSLYLQMNSLRAEDTAVYYCAREGILWFGDLPTFWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGGLPGCKWDLLIKQWVCDPLGSGSATGGSGSVASSGSGSATHLLPGCKWDLLIKQWVCDPL

SEQ ID NO: 18 - protein, 511 aa, synthetic construct
  REGION 1..511: note = source = /note="Description of Artificial Sequence: Syntheticpolypeptide"
EVQLVESGGGLVQPGGSLRLSCAASGFTFSSYWMSWVRQAPGKGLEWVAYIKQDGNEKYYVDSVKGRFTISRDNAKNSLYLQMNSLRAEDTAVYYCAREGILWFGDLPTFWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGGLPGCKWDLLIKQWVCDPLGSGSATGGSGSSASSGSGSATHLLPGCKWDLLIKQWVCDPL

SEQ ID NO: 19 - protein, 214 aa, synthetic construct [sequence omitted]

SEQ ID NO: 20 - skipped

SEQ ID NO: 21 - protein, 5 aa, synthetic construct
  REGION 1..5: note = source = /note="Description of Artificial Sequence: Syntheticpeptide"
LEWIG

SEQ ID NO: 22-23 - skipped (2 entries)

SEQ ID NO: 24 - protein, 23 aa, synthetic construct
  REGION 1..23: note = source = /note="Description of Artificial Sequence: Syntheticpeptide"
GSGSATGGSGSGASSGSGSATGS

SEQ ID NO: 25 - protein, 447 aa, synthetic construct
  REGION 1..447: note = source = /note="Description of Artificial Sequence: Syntheticpolypeptide"
EVQLVESGGGLVQPGGSLRLSCAASGFTFSSYWMSWVRQAPGKGLEWVAYIKQDGNEKYYVDSVKGRFTISRDNAKNSLYLQMNSLRAEDTAVYYCAREGILWFGDLPTFWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 26 - protein, 508 aa, synthetic construct
  REGION 1..508: note = source = /note="Description of Artificial Sequence: Syntheticpolypeptide"
LPGCKWDLLIKQWVCDPLGSGSATGGSGSGASSGSGSATGSLPGCKWDLLIKQWVCDPLGGEVQLVESGGGLVQPGGSLRLSCAASGFTFSSYWMSWVRQAPGKGLEWVAYIKQDGNEKYYVDSVKGRFTISRDNAKNSLYLQMNSLRAEDTAVYYCAREGILWFGDLPTFWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 27 - protein, 275 aa, synthetic construct
  REGION 1..275: note = source = /note="Description of Artificial Sequence: Syntheticpolypeptide"
LPGCKWDLLIKQWVCDPLGSGSATGGSGSGASSGSGSATGSLPGCKWDLLIKQWVCDPLGGDIQMTQSPSSLSASVGDRVTITCRASQGISNWLAWYQQKPEKAPKSLIYAASSLQSGVPSRFSGSGSGTDFTLTISSLQPEDFATYYCQQYDSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 511 aa, synthetic construct
  REGION 1..511: note = source = /note="Description of Artificial Sequence: Syntheticpolypeptide"
EVQLVESGGGLVQPGGSLRLSCAASGFTFSSYWMSWVRQAPGKGLEWVAYIKQDGNEKYYVDSVKGRFTISRDNAKNSLYLQMNSLRAEDTAVYYCAREGILWFGDLPTFWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGLPGCKWDLLIKQWVCDPLGGGGSGGGGSGGGGSGGGGSGGGGSLPGCKWDLLIKQWVCDPL

SEQ ID NO: 29 - protein, 510 aa, synthetic construct
  REGION 1..510: note = source = /note="Description of Artificial Sequence: Syntheticpolypeptide"
EVQLVESGGGLVQPGGSLRLSCAASGFTFSSYWMSWVRQAPGKGLEWVAYIKQDGNEKYYVDSVKGRFTISRDNAKNSLYLQMNSLRAEDTAVYYCAREGILWFGDLPTFWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMGGLPGCKWDLLIKQWVCDPLGSGSATGGSGSGASSGSGSATGSLPGCKWDLLIKQWVCDPLGGTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 30 - protein, 490 aa, synthetic construct
  REGION 1..490: note = source = /note="Description of Artificial Sequence: Syntheticpolypeptide"
EVQLVESGGGLVQPGGSLRLSCAASGFTFSSYWMSWVRQAPGKGLEWVAYIKQDGNEKYYVDSVKGRFTISRDNAKNSLYLQMNSLRAEDTAVYYCAREGILWFGDLPTFWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMGGLPGCKWDLLIKQWVCDPLGGTKNQVSLTCLVKGFYPSDIAVEWESNGQPENNYKTTPPMLDSDGSFFLYSKLTVDKSRWQQGNVFSCSVMHEALHNHYTQKSLSLSPGGGGGLPGCKWDLLIKQWVCDPL

SEQ ID NO: 31 - protein, 491 aa, synthetic construct
  REGION 1..491: note = source = /note="Description of Artificial Sequence: Syntheticpolypeptide"
EVQLVESGGGLVQPGGSLRLSCAASGFTFSSYWMSWVRQAPGKGLEWVAYIKQDGNEKYYVDSVKGRFTISRDNAKNSLYLQMNSLRAEDTAVYYCAREGILWFGDLPTFWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHGGLPGCKWDLLIKQWVCDPLGGEDPEVQFNWYVDGVEVHNAKTKPREEQFNSTFRVVSVLTVVHQDWLNGKEYKCKVSNKGLPAPIEKTISKTKGQPREPQVYTLPPSREEMGGLPGCKWDLLIKQWVCDPLGGTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 32 - protein, 293 aa, synthetic construct
  REGION 1..293: note = source = /note="Description of Artificial Sequence: Syntheticpolypeptide"
MLPGCKWDLLIKQWVCDPLGSGSATGGSGSTASSGSGSATHMLPGCKWDLLIKQWVCDPLGGGGGVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 228 aa, Homo sapiens
ERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 35 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 36 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 4 aa, synthetic construct
  REGION 1..4: note = source = /note="Description of Artificial Sequence: Syntheticpeptide"
GGGS

SEQ ID NO: 38 - protein, 4 aa, synthetic construct
  REGION 1..4: note = source = /note="Description of Artificial Sequence: Syntheticpeptide"
GGGP

SEQ ID NO: 39 - protein, 4 aa, synthetic construct
  REGION 1..4: note = source = /note="Description of Artificial Sequence: Syntheticpeptide"
GGGQ

SEQ ID NO: 40 - protein, 5 aa, synthetic construct
  REGION 1..5: note = source = /note="Description of Artificial Sequence: Syntheticpeptide"
GGGGG

SEQ ID NO: 41 - DNA, 54 bp, synthetic construct
  misc_feature 1..54: note = source = /note="Description of Artificial Sequence: Syntheticoligonucleotide"
  source 1..54: mol_type = other DNA
ctgccgggttgtaaatgggacctgctgatcaaacagtgggtttgtgacccgctg

SEQ ID NO: 42 - DNA, 12 bp, synthetic construct
  misc_feature 1..12: note = source = /note="Description of Artificial Sequence: Syntheticoligonucleotide"
  source 1..12: mol_type = other DNA
ggtggtggtggt

SEQ ID NO: 43 - DNA, 69 bp, synthetic construct
  modified_base 36: mod_base = OTHER; note = a, c, t, g, unknown or other
  misc_feature 1..69: note = source = /note="Description of Artificial Sequence: Syntheticoligonucleotide"
  source 1..69: mol_type = other DNA
ggatccggttctgctactggtggttccggctccdbngcaagctctggttcaggcagtgcgactcatctg

SEQ ID NO: 44 - DNA, 69 bp, synthetic construct
  misc_feature 1..69: note = source = /note="Description of Artificial Sequence: Syntheticoligonucleotide"
  source 1..69: mol_type = other DNA
ggatccggttctgctactggtggttccggctccgtcgcaagctctggttcaggcagtgcgactcatctg

SEQ ID NO: 45 - DNA, 69 bp, synthetic construct
  misc_feature 1..69: note = source = /note="Description of Artificial Sequence: Syntheticoligonucleotide"
  source 1..69: mol_type = other DNA
ggatccggttctgctactggtggttccggctcctcggcaagctctggttcaggcagtgcgactcatctg

SEQ ID NO: 46 - DNA, 33 bp, synthetic construct
  misc_feature 1..33: note = source = /note="Description of Artificial Sequence: Syntheticoligonucleotide"
  source 1..33: mol_type = other DNA
cgggcgagtcagggtattagcaactggttagcc

SEQ ID NO: 47 - DNA, 21 bp, synthetic construct
  misc_feature 1..21: note = source = /note="Description of Artificial Sequence: Syntheticoligonucleotide"
  source 1..21: mol_type = other DNA
gctgcatccagtttgcaaagt

SEQ ID NO: 48 - DNA, 27 bp, synthetic construct
  misc_feature 1..27: note = source = /note="Description of Artificial Sequence: Syntheticoligonucleotide"
  source 1..27: mol_type = other DNA
caacagtatgatagttaccctcggacg

SEQ ID NO: 49 - DNA, 15 bp, synthetic construct
  misc_feature 1..15: note = source = /note="Description of Artificial Sequence: Syntheticoligonucleotide"
  source 1..15: mol_type = other DNA
agttattggatgagt

SEQ ID NO: 50 - DNA, 51 bp, synthetic construct
  misc_feature 1..51: note = source = /note="Description of Artificial Sequence: Syntheticoligonucleotide"
  source 1..51: mol_type = other DNA
tacataaagcaagatggaaatgagaaatactatgtggactctgtgaagggc

SEQ ID NO: 51 - DNA, 36 bp, synthetic construct
  misc_feature 1..36: note = source = /note="Description of Artificial Sequence: Syntheticoligonucleotide"
  source 1..36: mol_type = other DNA
gaagggatactttggttcggggacttaccgacgttc

SEQ ID NO: 52 - DNA, 324 bp, synthetic construct
  misc_feature 1..324: note = source = /note="Description of Artificial Sequence: Syntheticpolynucleotide"
  source 1..324: mol_type = other DNA
gacatccagatgacccagtctccatcctcactgtctgcatctgtaggagacagagtcaccatcacttgtcgggcgagtcagggtattagcaactggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtatgatagttaccctcggacgttcggccaagggaccaaggtggaaatcaaacga

SEQ ID NO: 53 - DNA, 363 bp, synthetic construct
  misc_feature 1..363: note = source = /note="Description of Artificial Sequence: Syntheticpolynucleotide"
  source 1..363: mol_type = other DNA
gaggtgcagctggtggagtctgggggaggcttggtccagcctggggggtccctgagactctcctgtgcagcttctggatttacctttagtagttattggatgagttgggtccgccaggctccagggaaagggctggagtgggtggcctacataaagcaagatggaaatgagaaatactatgtggactctgtgaagggccgattcaccatctccagagacaacgccaagaactcattgtatctgcaaatgaacagcctgagagccgaggacacggctgtgtattactgtgcgagggaagggatactttggttcggggacttaccgacgttctggggccagggaaccctggtcaccgtctctagt

SEQ ID NO: 54 - DNA, 1527 bp, synthetic construct
  misc_feature 1..1527: note = source = /note="Description of Artificial Sequence: Syntheticpolynucleotide"
  source 1..1527: mol_type = other DNA
gaggtgcagctggtggagtctgggggaggcttggtccagcctggggggtccctgagactctcctgtgcagcttctggatttacctttagtagttattggatgagttgggtccgccaggctccagggaaagggctggagtgggtggcctacataaagcaagatggaaatgagaaatactatgtggactctgtgaagggccgattcaccatctccagagacaacgccaagaactcattgtatctgcaaatgaacagcctgagagccgaggacacggctgtgtattactgtgcgagggaagggatactttggttcggggacttaccgacgttctggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtaacggtgccctcctcaaatttcgggacgcagacatatacatgcaatgtggatcataagccttccaacacgaaggtggacaagactgtggagcggaagtgttgcgtcgagtgcccaccgtgtcccgctcctccggtcgctggcccatcagtatttctcttccctcccaagccaaaagatacactcatgatctcaagaaccccagaagtgacttgtgtggtcgtggacgtgtcgcatgaggatccggaggtgcagtttaactggtatgtggatggcgtagaagtccacaacgccaagaccaagcctagagaggaacaattcaactcgacgttcagggtggtcagcgtgttgacagtagtccaccaggactggcttaatgggaaggaatacaaatgtaaggtctcaaacaaagggctcccggcacccattgagaagacaatttccaaaaccaagggacagcccagggaaccccaagtgtatacgctgcccccaagccgggaggaaatgacgaaaaatcaggtcagcctcacgtgtctcgtaaagggattttacccgtcggacatcgcggtggagtgggagtcaaatggacagcccgaaaacaactataagaccacaccaccgatgctcgactccgacggaagcttctttttgtactcgaaactgacggtggacaaatcgcgctggcaacaggggaatgtctttagctgctcggtcatgcacgaggccctccacaatcattacactcagaaaagcttgtcgctctcgccgggtgggggtggaggactgcccggttgcaaatgggatctgttgatcaaacagtgggtatgcgaccctttgggaagcggctcggcgacgggtgggtcggggtcgggtgcgtccagcggatcgggctcggccactgggtcactccctggatgcaagtgggatcttcttatcaagcaatgggtgtgcgatcccctc

SEQ ID NO: 55 - DNA, 1533 bp, synthetic construct
  misc_feature 1..1533: note = source = /note="Description of Artificial Sequence: Syntheticpolynucleotide"
  source 1..1533: mol_type = other DNA
gaggtgcagctggtggagtctgggggaggcttggtccagcctggggggtccctgagactctcctgtgcagcttctggatttacctttagtagttattggatgagttgggtccgccaggctccagggaaagggctggagtgggtggcctacataaagcaagatggaaatgagaaatactatgtggactctgtgaagggccgattcaccatctccagagacaacgccaagaactcattgtatctgcaaatgaacagcctgagagccgaggacacggctgtgtattactgtgcgagggaagggatactttggttcggggacttaccgacgttctggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtaacggtgccctcctcaaatttcgggacgcagacatatacatgcaatgtggatcataagccttccaacacgaaggtggacaagactgtggagcggaagtgttgcgtcgagtgcccaccgtgtcccgctcctccggtcgctggcccatcagtatttctcttccctcccaagccaaaagatacactcatgatctcaagaaccccagaagtgacttgtgtggtcgtggacgtgtcgcatgaggatccggaggtgcagtttaactggtatgtggatggcgtagaagtccacaacgccaagaccaagcctagagaggaacaattcaactcgacgttcagggtggtcagcgtgttgacagtagtccaccaggactggcttaatgggaaggaatacaaatgtaaggtctcaaacaaagggctcccggcacccattgagaagacaatttccaaaaccaagggacagcccagggaaccccaagtgtatacgctgcccccaagccgggaggaaatgacgaaaaatcaggtcagcctcacgtgtctcgtaaagggattttacccgtcggacatcgcggtggagtgggagtcaaatggacagcccgaaaacaactataagaccacaccaccgatgctcgactccgacggaagcttctttttgtactcgaaactgacggtggacaaatcgcgctggcaacaggggaatgtctttagctgctcggtcatgcacgaggccctccacaatcattacactcagaaaagcttgtcgctctcgccgggtaaaggtggtggtggtggtctgccgggttgtaaatgggacctgctgatcaaacagtgggtttgtgacccgctgggatccggttctgctactggtggttccggctccgtcgcaagctctggttcaggcagtgcgactcatctgctgccgggttgtaaatgggacctgctgatcaaacagtgggtttgtgacccgctg

SEQ ID NO: 56 - DNA, 1533 bp, synthetic construct
  misc_feature 1..1533: note = source = /note="Description of Artificial Sequence: Syntheticpolynucleotide"
  source 1..1533: mol_type = other DNA
gaggtgcagctggtggagtctgggggaggcttggtccagcctggggggtccctgagactctcctgtgcagcttctggatttacctttagtagttattggatgagttgggtccgccaggctccagggaaagggctggagtgggtggcctacataaagcaagatggaaatgagaaatactatgtggactctgtgaagggccgattcaccatctccagagacaacgccaagaactcattgtatctgcaaatgaacagcctgagagccgaggacacggctgtgtattactgtgcgagggaagggatactttggttcggggacttaccgacgttctggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtaacggtgccctcctcaaatttcgggacgcagacatatacatgcaatgtggatcataagccttccaacacgaaggtggacaagactgtggagcggaagtgttgcgtcgagtgcccaccgtgtcccgctcctccggtcgctggcccatcagtatttctcttccctcccaagccaaaagatacactcatgatctcaagaaccccagaagtgacttgtgtggtcgtggacgtgtcgcatgaggatccggaggtgcagtttaactggtatgtggatggcgtagaagtccacaacgccaagaccaagcctagagaggaacaattcaactcgacgttcagggtggtcagcgtgttgacagtagtccaccaggactggcttaatgggaaggaatacaaatgtaaggtctcaaacaaagggctcccggcacccattgagaagacaatttccaaaaccaagggacagcccagggaaccccaagtgtatacgctgcccccaagccgggaggaaatgacgaaaaatcaggtcagcctcacgtgtctcgtaaagggattttacccgtcggacatcgcggtggagtgggagtcaaatggacagcccgaaaacaactataagaccacaccaccgatgctcgactccgacggaagcttctttttgtactcgaaactgacggtggacaaatcgcgctggcaacaggggaatgtctttagctgctcggtcatgcacgaggccctccacaatcattacactcagaaaagcttgtcgctctcgccgggtaaaggtggtggtggtggtctgccgggttgtaaatgggacctgctgatcaaacagtgggtttgtgacccgctgggatccggttctgctactggtggttccggctcctcggcaagctctggttcaggcagtgcgactcatctgctgccgggttgtaaatgggacctgctgatcaaacagtgggtttgtgacccgctg

SEQ ID NO: 57 - DNA, 642 bp, synthetic construct
  misc_feature 1..642: note = source = /note="Description of Artificial Sequence: Syntheticpolynucleotide"
  source 1..642: mol_type = other DNA
gacatccagatgacccagtctccatcctcactgtctgcatctgtaggagacagagtcaccatcacttgtcgggcgagtcagggtattagcaactggttagcctggtatcagcagaaaccagagaaagcccctaagtccctgatctatgctgcatccagtttgcaaagtggggtcccatcaaggttcagcggcagtggatctgggacagatttcactctcaccatcagcagcctgcagcctgaagattttgcaacttattactgccaacagtatgatagttaccctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 58 - DNA, 69 bp, synthetic construct
  misc_feature 1..69: note = source = /note="Description of Artificial Sequence: Syntheticoligonucleotide"
  source 1..69: mol_type = other DNA
ggaagcggctcggcgacgggtgggtcggggtcgggtgcgtccagcggatcgggctcggccactgggtca

SEQ ID NO: 59 - DNA, 1341 bp, synthetic construct
  misc_feature 1..1341: note = source = /note="Description of Artificial Sequence: Syntheticpolynucleotide"
  source 1..1341: mol_type = other DNA
gaggtgcagctggtggagtctgggggaggcttggtccagcctggggggtccctgagactctcctgtgcagcttctggatttacctttagtagttattggatgagttgggtccgccaggctccagggaaagggctggagtgggtggcctacataaagcaagatggaaatgagaaatactatgtggactctgtgaagggccgattcaccatctccagagacaacgccaagaactcattgtatctgcaaatgaacagcctgagagccgaggacacggctgtgtattactgtgcgagggaagggatactttggttcggggacttaccgacgttctggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtaacggtgccctcctcaaatttcgggacgcagacatatacatgcaatgtggatcataagccttccaacacgaaggtggacaagactgtggagcggaagtgttgcgtcgagtgcccaccgtgtcccgctcctccggtcgctggcccatcagtatttctcttccctcccaagccaaaagatacactcatgatctcaagaaccccagaagtgacttgtgtggtcgtggacgtgtcgcatgaggatccggaggtgcagtttaactggtatgtggatggcgtagaagtccacaacgccaagaccaagcctagagaggaacaattcaactcgacgttcagggtggtcagcgtgttgacagtagtccaccaggactggcttaatgggaaggaatacaaatgtaaggtctcaaacaaagggctcccggcacccattgagaagacaatttccaaaaccaagggacagcccagggaaccccaagtgtatacgctgcccccaagccgggaggaaatgacgaaaaatcaggtcagcctcacgtgtctcgtaaagggattttacccgtcggacatcgcggtggagtgggagtcaaatggacagcccgaaaacaactataagaccacaccaccgatgctcgactccgacggaagcttctttttgtactcgaaactgacggtggacaaatcgcgctggcaacaggggaatgtctttagctgctcggtcatgcacgaggccctccacaatcattacactcagaaaagcttgtcgctctcgccgggtaaa

SEQ ID NO: 60 - DNA, 1524 bp, synthetic construct
  misc_feature 1..1524: note = source = /note="Description of Artificial Sequence: Syntheticpolynucleotide"
  source 1..1524: mol_type = other DNA
cttcccggatgcaagtgggatctgttgatcaagcaatgggtctgcgaccctctcgggtcagggtccgcgaccggtggatcggggtcgggagcgtcatcgggcagcggaagcgctacgggatcacttcccgggtgcaaatgggacctcctgatcaaacaatgggtatgtgatccgctcggtggcgaggtgcagctggtggagtctgggggaggcttggtccagcctggggggtccctgagactctcctgtgcagcttctggatttacctttagtagttattggatgagttgggtccgccaggctccagggaaagggctggagtgggtggcctacataaagcaagatggaaatgagaaatactatgtggactctgtgaagggccgattcaccatctccagagacaacgccaagaactcattgtatctgcaaatgaacagcctgagagccgaggacacggctgtgtattactgtgcgagggaagggatactttggttcggggacttaccgacgttctggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 61 - DNA, 825 bp, synthetic construct
  misc_feature 1..825: note = source = /note="Description of Artificial Sequence: Syntheticpolynucleotide"
  source 1..825: mol_type = other DNA
ctccctgggtgcaaatgggacctgttgattaagcagtgggtctgcgaccctctcggatcgggaagcgcaactgggggttcaggctcaggggctagctccggatcggggtcggccacagggtcgctccccggatgtaagtgggaccttttgattaaacagtgggtgtgcgatccacttggaggtgatatccagatgacacagtcaccctcgtcgttgagcgccagcgtgggagatagagtgacgatcacctgtcgagccagccagggcatctccaactggcttgcgtggtaccaacaaaagcccgagaaggcaccgaaatcgctgatctacgcggcgtcgtcactgcagtcgggtgtaccgtcgcggtttagcgggtccgggtccggaacggacttcacgctcacgatttcctcattgcagccggaagattttgcgacttattactgtcagcaatatgactcatatccccgcacattcggtcagggaaccaaggtcgagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 62 - DNA, 1533 bp, synthetic construct
  misc_feature 1..1533: note = source = /note="Description of Artificial Sequence: Syntheticpolynucleotide"
  source 1..1533: mol_type = other DNA
gaggtgcagctggtggagtctgggggaggcttggtccagcctggggggtccctgagactctcctgtgcagcttctggatttacctttagtagttattggatgagttgggtccgccaggctccagggaaagggctggagtgggtggcctacataaagcaagatggaaatgagaaatactatgtggactctgtgaagggccgattcaccatctccagagacaacgccaagaactcattgtatctgcaaatgaacagcctgagagccgaggacacggctgtgtattactgtgcgagggaagggatactttggttcggggacttaccgacgttctggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtaacggtgccctcctcaaatttcgggacgcagacatatacatgcaatgtggatcataagccttccaacacgaaggtggacaagactgtggagcggaagtgttgcgtcgagtgcccaccgtgtcccgctcctccggtcgctggcccatcagtatttctcttccctcccaagccaaaagatacactcatgatctcaagaaccccagaagtgacttgtgtggtcgtggacgtgtcgcatgaggatccggaggtgcagtttaactggtatgtggatggcgtagaagtccacaacgccaagaccaagcctagagaggaacaattcaactcgacgttcagggtggtcagcgtgttgacagtagtccaccaggactggcttaatgggaaggaatacaaatgtaaggtctcaaacaaagggctcccggcacccattgagaagacaatttccaaaaccaagggacagcccagggaaccccaagtgtatacgctgcccccaagccgggaggaaatgacgaaaaatcaggtcagcctcacgtgtctcgtaaagggattttacccgtcggacatcgcggtggagtgggagtcaaatggacagcccgaaaacaactacaaaacgaccccacctatgctcgattcggacggcagcttctttttgtattcaaagttgacagtggacaaatcgcgatggcagcagggcaacgtcttctcatgttcagtaatgcatgaggcccttcacaaccactacacgcagaagtccctctcattgtcgccgggtgggggtggaggactgcccgggtgcaagtgggacctcttgatcaaacagtgggtatgcgaccctttgggagggggtgggtcaggagggggaggttccggtggaggtggttccgggggaggcggatcaggaggtggaggatcgttgcccggctgtaagtgggatctgctgatcaagcagtgggtctgtgatcctttg

SEQ ID NO: 63 - DNA, 1530 bp, synthetic construct
  misc_feature 1..1530: note = source = /note="Description of Artificial Sequence: Syntheticpolynucleotide"
  source 1..1530: mol_type = other DNA
gaggtgcagctggtggagtctgggggaggcttggtccagcctggggggtccctgagactctcctgtgcagcttctggatttacctttagtagttattggatgagttgggtccgccaggctccagggaaagggctggagtgggtggcctacataaagcaagatggaaatgagaaatactatgtggactctgtgaagggccgattcaccatctccagagacaacgccaagaactcattgtatctgcaaatgaacagcctgagagccgaggacacggctgtgtattactgtgcgagggaagggatactttggttcggggacttaccgacgttctggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgccaccttcgcgggaggaaatgggaggactccccgggtgcaagtgggatcttcttatcaaacagtgggtatgcgacccgctggggtcagggtcagcgacaggtggatcgggtagcggcgcatcgagcggatcagggtccgcgacgggctcacttcccggatgcaaatgggacctcttgattaagcagtgggtgtgtgacccgttgggtggaacgaagaatcaggtctcgttgacgtgtctggtgaaggggttttatccctcggatatcgctgtcgagtgggagtcgaatggacagcccgaaaacaactacaagaccaccccgcctatgctggactccgatggttccttctttttgtactcgaaactgactgtggataagagcaggtggcagcaagggaatgtattctcgtgttccgtcatgcacgaagccctccataaccactatacacaaaaatcgctttcacttagcccgggaaaa

SEQ ID NO: 64 - DNA, 1470 bp, synthetic construct
  misc_feature 1..1470: note = source = /note="Description of Artificial Sequence: Syntheticpolynucleotide"
  source 1..1470: mol_type = other DNA
gaggtgcagctggtggagtctgggggaggcttggtccagcctggggggtccctgagactctcctgtgcagcttctggatttacctttagtagttattggatgagttgggtccgccaggctccagggaaagggctggagtgggtggcctacataaagcaagatggaaatgagaaatactatgtggactctgtgaagggccgattcaccatctccagagacaacgccaagaactcattgtatctgcaaatgaacagcctgagagccgaggacacggctgtgtattactgtgcgagggaagggatactttggttcggggacttaccgacgttctggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgccgccctcgagagaagagatgggcgggttgccggggtgtaagtgggacttgctgattaaacaatgggtgtgcgaccctctgggcggtaccaagaatcaggtctcactgacatgtctcgtaaaaggtttttacccgtcagatatcgcggtcgagtgggaatccaacggacaacccgagaataactacaagacgactcccccaatgctcgattcggatggatccttcttcctttatagcaaacttacagtagacaaatcacggtggcagcaggggaacgtgtttagctgttcggtgatgcacgaagccttgcataatcactatacgcagaagtcgctttccctgtcgccgggagggggaggtgggctccctggatgcaagtgggatcttttgatcaagcagtgggtctgcgaccccctc

SEQ ID NO: 65 - DNA, 1473 bp, synthetic construct
  misc_feature 1..1473: note = source = /note="Description of Artificial Sequence: Syntheticpolynucleotide"
  source 1..1473: mol_type = other DNA
gaggtgcagctggtggagtctgggggaggcttggtccagcctggggggtccctgagactctcctgtgcagcttctggatttacctttagtagttattggatgagttgggtccgccaggctccagggaaagggctggagtgggtggcctacataaagcaagatggaaatgagaaatactatgtggactctgtgaagggccgattcaccatctccagagacaacgccaagaactcattgtatctgcaaatgaacagcctgagagccgaggacacggctgtgtattactgtgcgagggaagggatactttggttcggggacttaccgacgttctggggccagggaaccctggtcaccgtctctagtgcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggatgtaagccatgggggactgcctggatgcaagtgggatcttctcattaagcaatgggtctgtgaccctttgggcggagaggacccggaagtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgccgccctcgagagaagagatgggcgggttgccggggtgtaagtgggacttgctgattaaacaatgggtgtgcgaccctctgggcggtaccaagaatcaggtctcactgacatgtctcgtaaaaggtttttacccgtcagatatcgcggtcgagtgggaatccaacggacaacccgagaataactacaagacgactcccccaatgctcgattcggatggatccttcttcctttatagcaaacttacagtagacaaatcacggtggcagcaggggaacgtgtttagctgttcggtgatgcacgaagccttgcataatcactatacgcagaagtcgctttccctgtctccgggtaaa

SEQ ID NO: 66 - DNA, 879 bp, synthetic construct
  misc_feature 1..879: note = source = /note="Description of Artificial Sequence: Syntheticpolynucleotide"
  source 1..879: mol_type = other DNA
atgcttccaggttgtaaatgggatcttcttattaaacaatgggtttgtgatccacttggttctggttctgctactggtggttccggctccaccgcaagctctggttcaggttctgctactcatatgctgccgggttgtaaatgggacctgctgatcaaacagtgggtttgtgacccgctgggtggaggcggtggggtcgacaaaactcacacatgtccaccttgtccagctccggaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 67 - DNA, 696 bp, Homo sapiens
  source 1..696: mol_type = unassigned DNA
gagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 68 - DNA, 684 bp, Homo sapiens
  source 1..684: mol_type = unassigned DNA
gagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 69 - DNA, 837 bp, Homo sapiens
  source 1..837: mol_type = unassigned DNA
gagctcaaaaccccacttggtgacacaactcacacatgcccacggtgcccagagcccaaatcttgtgacacacctcccccgtgcccacggtgcccagagcccaaatcttgtgacacacctcccccgtgcccacggtgcccagagcccaaatcttgtgacacacctcccccatgcccacggtgcccagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagttcaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 70 - DNA, 687 bp, Homo sapiens
  source 1..687: mol_type = unassigned DNA
gagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 71 - protein, 25 aa, synthetic construct
  REGION 1..25: note = source = /note="Description of Artificial Sequence: Syntheticpeptide"
GGGGSGGGGSGGGGSGGGGSGGGGS

SEQ ID NO: 72 - protein, 5 aa, synthetic construct
  REGION 1..5: note = source = /note="Description of Artificial Sequence: Syntheticpeptide"
GGGGS

(19) United States
(12) Patent Application Publication
(10) Pub. No.: US 2026/0132222 A1
(43) Pub. Date: May 14, 2026

(54) PROTEINS SPECIFIC FOR BAFF AND B7RP1 AND USES THEREOF

(71) Applicant: AMGEN INC., Thousand Oaks, CA (US)

(72) Inventors: Hailing HSU, Moorpark, CA (US); Ming ZHANG, Thousand Oaks, CA (US); Gunasekaran KANNAN, Daly City, CA (US); Frederick W. JACOBSEN, Newbury Park, CA (US); Wayne H. TSUJI, Seattle, WA (US)

(73) Assignee: AMGEN INC., Thousand Oaks, CA (US)

(21) Appl. No.: 19/371,657

(22) Filed: Oct. 28, 2025

Related U.S. Application Data
(62) Division of application No. 17/936,062, filed on Sep. 28, 2022, now Pat. No. 12,479,930.
Application No. 17/936,062 is a continuation of application No. 16/579,321, filed on Sep. 23, 2019, now Pat. No. 11,492,417.
Application No. 16/579,321 is a continuation of application No. 14/776,399, now Pat. No. 10,421,823.
(60) Provisional application No. 61/780,260, filed on Mar. 13, 2013.
(60) Provisional application No. 61/942,776, filed on Feb. 21, 2014.

(51) Int. Cl. C07K 16/46; A61K 39/00; A61K 39/395; A61P 19/02; A61P 29/02; A61P 37/06; C07K 16/18; C07K 16/24; C07K 16/28

(52) U.S. Cl. CPC C07K 16/468; A61K 39/3955; A61P 19/02; A61P 29/02; A61P 37/06; C07K 16/18; C07K 16/241; C07K 16/2827; C07K 16/2875; A61K 2039/505; C07K 2317/31; C07K 2317/524; C07K 2317/526; C07K 2317/565; C07K 2317/73; C07K 2317/76; C07K 2317/90; C07K 2317/92; C07K 2317/94; C07K 2319/00

(57) ABSTRACT

Described herein are bispecific proteins specific for BAFF and B7RP1, nucleic acids encoding such proteins, methods of making such proteins, and uses for such proteins.

### CROSS-REFERENCE TO RELATED APPLICATIONS

This application is a divisional of U.S. application Ser. No. 17/936,062, filed Sep. 28, 2022; which is a continuation of U.S. application Ser. No. 16/579,321, filed Sep. 23, 2019, now U.S. Pat. No. 11,492,417; which is a continuation of U.S. application Ser. No. 14/776,399, filed Sep. 14, 2015, now U.S. Pat. No. 10,421,823; which is the U.S. National Phase of International Patent Application No. PCT/US2014/024908, filed Mar. 12, 2014; which claims the benefit of U.S. Provisional Application Nos. 61/780,260, filed Mar. 13, 2013, and 61/942,776, filed Feb. 21, 2014; each of which are incorporated herein in their entirety.

### REFERENCE TO SEQUENCE LISTING SUBMITTED ELECTRONICALLY

The present application contains a Sequence Listing, which has been submitted electronically in XML format and is hereby incorporated by reference in its entirety. The computer readable format copy of the Sequence Listing, which was created Oct. 22, 2025, is named A-1887-US05-DIV_Seqlisting and is 118,700 bytes in size.

### FIELD

The bispecific molecules described herein are within the field of protein therapeutics.

### BACKGROUND

Most therapeutic proteins bind to a single target protein with high specificity, thereby interfering with the activity of this single target protein. That protein may be a part of one or more biological pathways that mediate a human disease being treated, and the therapeutic protein may therefore inhibit disease progression. However, efficacy of therapeutic proteins is rarely complete for all patients. Incomplete efficacy of therapeutic proteins could be due in some cases to the complexity of a disease. For example, some diseases may be mediated by multiple biological pathways, or different biological pathways may play a predominant role in mediating disease activity in different patients having the same clinically-defined condition. Hence, in some diseases it may be advantageous to simultaneously inhibit at least two biological pathways.

### SUMMARY

Herein is provided a bispecific protein that can bind to and inhibit the biological activity of both human B7-related protein 1 (B7RP1, also known as GL50 and T-cell co-stimulator ligand (ICOSLG)) and human B-cell activating factor (BAFF, also known as tumor necrosis factor superfamily, member 13b (TNFSF13B)). BAFF plays a role in B cell survival, and B7RP1 plays a role in T cell costimulation. Thus, a protein that inhibits the activity of both proteins interferes with the activity of both B and T cells.

Described herein is bispecific protein, wherein the protein can inhibit BAFF-mediated proliferation of human B cells and wherein the protein can inhibit B7RP1-mediated proliferation of human T cells. The bispecific protein can comprise an IgG antibody comprising two immunoglobulin heavy chains having different amino acid sequences and two immunoglobulin light chains having different amino acid sequences. The IgG antibody can inhibit BAFF-mediated proliferation of human B cells and B7RP1-mediated proliferation of human T cells The IgG antibody can be an IgG1, IgG2, IgG3, or IgG4 antibody and can be a human or humanized IgG antibody. The bispecific protein can comprise a light chain complementarity determining region 1 (CDR1) comprising the amino acid sequence of SEQ ID NO:8, a light chain complementarity determining region 2 (CDR2) comprising the amino acid sequence of SEQ ID NO:9, a light chain complementarity determining region 3 (CDR3) comprising the amino acid sequence of SEQ ID NO:10, a heavy chain CDR1 comprising the amino acid sequence of SEQ ID NO:11, a heavy chain CDR2 comprising the amino acid sequence of SEQ ID NO:12, and a heavy chain CDR3 comprising the amino acid sequence of SEQ ID NO:13. Further, the bispecific protein can comprise a heavy chain variable region comprising SEQ ID NO:15 or a variant thereof and a light chain variable region comprising SEQ ID NO:14 or a variant thereof. Such variant sequences can comprise not more than 10 deletions, insertions of substitutions of a single amino acid per 100 amino acids relative to a reference sequence.

In an alternate embodiment, a bispecific protein that can inhibit BAFF-mediated proliferation of human B cells and that can inhibit B7RP1-mediated proliferation of human T cells can comprise: (a) a polypeptide comprising an amino acid sequence having the following formula: A-L1-P-L2-P, wherein A is an immunoglobulin heavy chain of an IgG antibody, L1 is a first linker of that is absent or is 3 to 40 amino acids long, P is a BAFF-binding peptide that is 10 to 40 amino acids long, and L2 is a peptide linker that is absent or is 5 to 50 amino acids long; and (b) an immunoglobulin light chain. The immunoglobulin heavy chain of (a) and the immunoglobulin light chain of (b) can form an IgG antibody, comprising two molecules of the polypeptide of (a) and two molecules of the light chain of (b), that can bind B7RP1 and/or can inhibit B7RP1-mediated proliferation of human T cells. The immunoglobulin heavy chain may be missing a lysine at its C-terminal end just upstream of L1. The IgG antibody can be a human or humanized IgG1, IgG2, IgG3, or IgG4 antibody. The BAFF-binding peptide P can have the amino acid sequence of SEQ ID NO:1, SEQ ID NO:2, or SEQ ID NO:3. L1 can have the amino acid sequence of SEQ ID NO:4, 37, 38, 39, or 40. L2 can have the amino acid sequence of SEQ ID NO: 5, 6, or 7. The bispecific protein can comprise a light chain CDR1 comprising the amino acid sequence of SEQ ID NO:8 (RASQGISNWLA), a light chain CDR2 comprising the amino acid sequence of SEQ ID NO:9 (AASSLQS), a light chain CDR3 comprising the amino acid sequence of SEQ ID NO:10 (QQYDSYPRT), a heavy chain CDR1 comprising the amino acid sequence of SEQ ID NO:11 (SYWMS), a heavy chain CDR2 comprising the amino acid sequence of SEQ ID NO:12 (YIKQDGNEKYYVDSVKG), and a heavy chain CDR3 comprising the amino acid sequence of SEQ ID NO:13 (EGILWFGDLPTF). The bispecific protein can comprise an immunoglobulin light chain variable region comprising the amino acid sequence of SEQ ID NO:14 and/or an immunoglobulin heavy chain variable region comprising the amino acid sequence of SEQ ID NO:15. The bispecific protein can comprise the amino acid sequence of SEQ ID NO:19 or a variant thereof and the amino acid sequence of SEQ ID NO:17 or 18 or variants thereof. Such variant sequences can comprise not more than 10 deletions, insertions of substitutions of a single amino acid per 100 amino acids relative to the reference sequence.

In a further aspect, herein is described a bispecific protein comprising: (a) a polypeptide comprising the amino acid sequence of SEQ ID NO:17 or SEQ ID NO:18 or variants thereof; and (b) another polypeptide comprising the amino acid sequence of SEQ ID NO:19 or a variant thereof. Such variant sequences can comprise not more than 10 deletions, insertions of substitutions of a single amino acid per 100 amino acids relative to the reference sequence. The bispecific protein can inhibit BAFF-mediated proliferation of human B cells and B7RP1-mediated proliferation of human T cells. The bispecific protein can be a tetramer comprising two molecules of the polypeptide of (a) and two molecules of the polypeptide of (b).

In another embodiment, herein is provided a protein comprising a linker comprising the amino acid sequence of SEQ ID NO:6 or SEQ ID NO:7. In some embodiments, this protein can inhibit BAFF-mediated proliferation of human B cells and/or B7RP1-mediated proliferation of human T cells. Such a protein can comprise the amino acid sequences of SEQ ID NO:1, SEQ ID NO:14, and/or SEQ ID NO:15. Such a protein can comprise an amino acid sequence comprising at least two copies of SEQ ID NO:1 separated by SEQ ID NO:6 or 7. In a further embodiment, such a protein can include an immunoglobulin light chain and an immunoglobulin heavy, and SEQ ID NO:6 or 7 can be downstream from the C-terminus of the heavy chain. In such embodiments, SEQ ID NO:6 or 7 can be flanked by peptides that bind to a protein other than that bound by the heavy and light chains.

Further, herein is described a pharmaceutical composition comprising any of the bispecific proteins herein described or the protein comprising the amino acid sequence of SEQ ID NO:6 or 7 and a physiologically acceptable excipient.

Also described herein is a nucleic acid encoding any polypeptide included in one of bispecific proteins or the proteins comprising SEQ ID NO:6 or SEQ ID NO:7 herein described. Exemplary nucleic acids encoding a polypeptide included in a bispecific protein include, for example, SEQ ID NOs: 55, 56, 60, 61, 62, and 63, among others. Vectors comprising such nucleic acids and host cells containing such vectors and/or nucleic acids are described. Further described herein is method for making a bispecific protein comprising culturing the host cell containing a nucleic acid encoding any of the bispecific proteins described herein under conditions such that the nucleic acid is expressed and recovering the protein from the cell mass or the culture medium. The host cell can be a mammalian cell, for example, a CHO cell, or a bacterial cell such as Escherichia coli.

In another aspect, described herein is a method for treating systemic lupus erythematosus, including lupus nephritis, comprising administering to a patient a therapeutically effective amount of any of the bispecific proteins described herein or a pharmaceutical composition comprising such a bispecific protein. Another therapeutic can be administered to the patient before, after, or concurrently with the bispecific protein. The other therapeutic can be a corticosteroid, an antimalarial, retinoic acid, an NSAID, cyclophosphamide, dehydroepiandrosterone, mycophenolate mofetil, azathioprine, chlorambucil, methotrexate, tacrolimus, dapsone, thalidomide, leflunomide, or cyclosporine.

In a further aspect, herein is described a method of treatment comprising administering to a patient a therapeutically effective amount of any of the bispecific proteins described herein or a pharmaceutical composition comprising a bispecific protein described herein, wherein the patient has a disease selected from the group consisting of: ANCA-positive vasculitis, rheumatoid arthritis (RA), Crohn's disease, ulcerative colitis, celiac disease, pemphigus, pemphigoid, subacute cutaneous lupus erythematosus (SCLE), multiple sclerosis, chronic inflammatory demyelinating polyneuropathy (CIDP), myasthenia gravis, Goodpasture's syndrome, glomerulonephritis, autoimmune hemolytic anemia (AIHA), idiopathic thrombocytopenia purpura (ITP), chronic active hepatitis, primary biliary cirrhosis, Sjogren's syndrome, systemic sclerosis, Hashimoto's thyroiditis, Graves' disease, Addison's disease, and multiple endocrine neoplasia (MEN).

In another aspect, herein is described a pharmaceutical composition comprising any of the bispecific proteins or the proteins comprising SEQ ID NO:6 or SEQ ID NO:7 herein described. The pharmaceutical composition can be, for example, for the treatment of systemic lupus erythematosus or lupus nephritis.

In another aspect, the use of any of the bispecific proteins provided herein as a medicament is described.

### BRIEF DESCRIPTION OF THE FIGURES

FIG. 1: Diagrams of bispecific proteins that bind to BAFF and B7RP1. Across the top row are listed the identifier for each construct. Across the second row is a brief descriptive phrase relating to the structure of each construct. Across the bottom row is a diagram of the structure of each construct. The unfilled ovals represent constant regions of an immunoglobulin heavy or light chain. The ovals filled with horizontal lines represent immunoglobulin heavy or light chain variable (VH or VL) regions. The small, solidly filled squares and loops represent BAFF-binding peptides. The hinge regions are shown as heavy vertical lines, while the disulfide bridges are shown as heavy horizontal lines. The sequence of “G4S” in FIG. 1 is disclosed in SEQ ID NO: 72.

FIGS. 2A-2B: Activity of bispecific proteins in a human B cell proliferation assay. The data shown in FIGS. 2A (top) and 2B (bottom) are from B cell proliferation assays performed as described in Example 1. In both panels, the x axis indicates the concentration (log [nM]) of the bispecific protein contained in the assay mixture, and the y axis indicates the amount of 3H-thymidine uptake (counts per minute (cpm)). The meaning of each symbol is indicated by an identifier for each protein assayed. Meanings of the identifiers are shown in FIG. 1 and explained in Example 1.

FIG. 3: Activity of bispecific proteins in a human T cell proliferation assay. The data shown is from T cell proliferation assays performed as described in Example 1. The x axis indicates the concentration (log [nM]) of the bispecific or αB7RP1 antibody in the assay mixture, and the y axis indicates percent of T cell 3H-thymidine uptake in the presence of B7RP1 inhibitors at the indicated concentrations relative to T cell 3H-thymidine uptake without B7RP1 inhibitors (percent of control). The identifier for each protein tested is indicated.

FIG. 4: Cytokine release by human tonsil cells stimulated with Staphylococcus enterotoxin B (SEB). Methods are described in Example 1. The y axes show the levels of signal detected for the various cytokines measured using Meso Scale Discovery (Rockville, Maryland) kits according to the manufacturer's instructions. The cells were treated with either αB7RP1 (lane 1), P74293 (lane 2), CTLA4-Ig (lane 3), or human IgG (lane 4). The cytokines assayed are indicated in the figure.

FIG. 5: Pharmacokinetic profile of bispecific constructs in mice. Methods for assessing the in vivo pharmacokinetic properties of P71617, P71619, P71621, P71622, P74293, and P74294 in mice are described in Example 1. As explained in Example 1, the bispecific proteins were detected by two different assays, one of which detected only the Fc portion of the proteins (data points indicated by filled diamonds; Fc assay) and one of which detected both the Fc and BAFF-binding portion of the proteins (data points indicated by filled squares; intact assay). The x axis indicates the time post injection (hours), and the y axis indicates the concentration of the protein detected in serum (ng/ml). The construct injected is indicated in each panel.

FIG. 6A: Inhibition of murine B cell proliferation by a murine surrogate bispecific molecule (the “murine surrogate”) that binds to BAFF and B7RP1. The assay was performed as described in Example 2. The murine surrogate comprises an anti-murine B7RP1 IgG antibody that has two copies of a BAFF-binding peptide attached to the C terminus of the immunoglobulin heavy chain of the antibody, as explained in Example 2. The positive control was a BAFF-binding peptibody (“αBAFF”). Data from the murine surrogate and αBAFF are indicated, respectively, by solidly filled circles and squares. The x axis indicates the concentration of these test proteins in the assay (log [pM]), and the y axis indicates 3H-thymidine incorporation (cpm).

FIG. 6B: Inhibition of B7RP1 binding to murine T cells by the murine surrogate. The assay was performed as described in Example 2. An anti-murine B7RP1 IgG antibody (“anti-mB7RP1”) was used as a positive control. Data from the murine surrogate and anti-mB7RP1 are indicated, respectively, by solidly filled circles and squares. The x axis indicates the concentration of these test proteins in the assay (log [pM]), and the y axis indicates the percent of murine B7RP1-Fc bound to the T cells.

FIG. 7: In vivo effects on immunological parameters of administration of sheep red blood cells to mice. All results shown in this figure are from assays described in Example 2. The proteins that the mice were treated with are indicated by the fill in each bar as follows: unfilled, anti-mB7RP1; vertical lines, αBAFF; horizontal lines, anti-mB7RP1 plus αBAFF; diagonal lines, the murine surrogate; checkerboard, mIgG1; and solid fill (in bottom panel only), mice not challenged with SBRC. Top panel, percentage of spleen B cells in mice challenged with sheep red blood cells (SRBC). The y axis indicates the percent of cells from the spleen that are B cells. Middle panel, percentage of spleen CD4⁺ T cells that are memory T cells in mice challenged with SRBC. Bottom panel, levels of anti-SRBC antibodies in serum from mice challenged with SRBC.

FIG. 8A: Proteinuria in NZB/NZW mice treated with various proteins. Methods are described in Example 2. The treatment for each group of mice is indicated as follows: filled circles, phosphate buffered saline (PBS); filled squares, murine IgG1 (an isotype control; 5 mg/kg); unfilled squares, anti-mB7RP1 (4.68 mg/kg); filled, upward-pointing triangles, αBAFF (1.88 mg/kg); unfilled, upward-pointing triangles, αBAFF (1.88 mg/kg) plus anti-mB7RP1 (4.68 mg/kg); and unfilled, downward-pointing triangles, the murine surrogate (5 mg/kg). The x axis indicates the age of the mice (months), and the y axis indicates the percent of mice that exhibited proteinuria, i.e., ≥300 mb/dL protein in urine.

FIG. 8B: Levels of antibodies against double stranded DNA (dsDNA) in NZB/NZW mice at 8.5 months of age treated with various proteins. Methods are described in Example 2. The x axis indicates the identity of the molecule(s) that the mice were treated with as follows: 1, anti-mB7RP1 (4.68 mg/kg); 2, αBAFF (1.88 mg/kg); 3, αBAFF (1.88 mg/kg) plus anti-mB7RP1 (4.68 mg/kg); 4, the murine surrogate bispecific (5 mg/kg); and 5, mIgG1 (the isotype control; 5 mg/kg). The y axis indicates the levels of anti-dsDNA antibodies detected as a percentage of the positive control. Each dot indicates data from a single mouse.

FIG. 9A: Levels of anti-dsDNA IgG in NZB/NZW mice. Methods are described in Example 2. Data from various groups of mice are identified as follows: 1, mice that received anti-mB7RP1 (14 mg/kg); 2, mice that received αBAFF (5.6 mg/kg); 3, mice that received a combination of anti-mB7RP1 (14 mg/kg) and αBAFF (5.6 mg/kg); 4, mice that received the murine surrogate (15 mg/kg); 5, mice that received the mIgG isotype control (15 mg/kg); and 6, mice that received PBS. The asterisks above lanes 1, 3, and 4 indicate a significant (*, p<0.05; ***, p<0.0001) difference between data in those lanes and data from lane 5 (mIgG).

FIG. 9B: Percentages of NZB/W F₁ mice in each group having proteinuria. Methods are described in Example 2. Data from various groups of mice are identified as follows: unfilled squares, mice that received anti-mB7RP1 (14 mg/kg); filled, upward-pointing triangles, mice that received αBAFF (5.6 mg/kg); unfilled, upward-pointing triangles, mice that received a combination of anti-mB7RP1 (14 mg/kg) and αBAFF (5.6 mg/kg); unfilled, downward-pointing triangles, mice that received the murine surrogate (15 mg/kg); filled squares, mice that received the mIgG isotype control (15 mg/kg); and filled circles, mice that received PBS. Significant differences were detected between the murine surrogate versus anti-mB7RP1 (p<0.01), αBAFF (p<0.0001), and mIgG (p<0.0001). The time window in which treatment occurred is indicated.

FIG. 10: Kidney scores of NZB/W F₁ mice. As explained in Example 2, kidneys were harvested when a mouse died, if that happened before the end of the study, or at the end of the study. Kidney scores were determined as described in Example 2, with higher scores indicating more severe kidney disease. Shown are averages for each group of mice plus appropriate error bars. The groups of mice received the following treatments: 1) anti-mB7RP1 (14 mg/kg), bar filled with vertical lines; 2) αBAFF (5.6 mg/kg), bar filled with horizontal lines; 3) combination of anti-mB7RP1 (14 mg/kg) and αBAFF (5.6 mg/kg), bar filled with windowpane checks; 4) the murine surrogate (15 mg/kg), bar filled with checkerboard pattern; 5) mIgG (15 mg/kg), bar filled with white dots on a black background; and 6) PBS, solidly filled bar. Asterisks indicate a significant difference from mice treated with mIgG with a p value of <0.05 (*) or <0.001 (***).

FIG. 11: Effects of inhibition of BAFF and/or B7RP1 on murine collagen-induced arthritis. Methods are described in Example 4. The five groups of mice were treated with test substances indicated as follows: mIgG, filled squares connected by solid lines; PBS, filled squares connected by dashed lines; anti-mB7RP1, filled circles connected by dashed lines; αBAFF, open circles connected by solid lines; and combination of anti-mB7RP1 and αBAFF, filled circles connected by solid lines. The top panel shows the percent incidence of arthritis of the various groups, and the bottom panel shows the average arthritic scores of the groups. The vertical, downward-pointing arrow in each panel indicates the time of the second immunization with bovine collagen.

BRIEF DESCRIPTION OF THE SEQUENCE LISTINGS

| SEQUENCE | |
|---|---|
| LISTING | |
| NUMBER | DESCRIPTION |
| SEQ ID NO: 1 | Amino acid sequence of a BAFF-binding peptide |
| SEQ ID NO: 2 | Amino acid sequence of a BAFF-binding peptide |
| SEQ ID NO: 3 | Amino acid sequence of a BAFF-binding peptide |
| SEQ ID NO: 4 | Amino acid sequence of a linker |
| SEQ ID NO: 5 | Amino acid sequence of a linker |
| SEQ ID NO: 6 | Amino acid sequence of a linker |
| SEQ ID NO: 7 | Amino acid sequence of a linker |
| SEQ ID NO: 8 | Amino acid sequence of a light chain CDR1 |
| SEQ ID NO: 9 | Amino acid sequence of a light chain CDR2 |
| SEQ ID NO: 10 | Amino acid sequence of a light chain CDR3 |
| SEQ ID NO: 11 | Amino acid sequence of a heavy chain CDR1 |
| SEQ ID NO: 12 | Amino acid sequence of a heavy chain CDR2 |
| SEQ ID NO: 13 | Amino acid sequence of a heavy chain CDR3 |
| SEQ ID NO: 14 | Amino acid sequence of a light chain variable region |
| SEQ ID NO: 15 | Amino acid sequence of a heavy chain variable region |
| SEQ ID NO: 16 | Amino acid sequence of a heavy chain of the P71619 |
| | BAFF/B7RP1 bispecific molecule |
| SEQ ID NO: 17 | Amino acid sequence of a heavy chain of the P74293 |
| | BAFF/B7RP1 bispecific molecule |
| SEQ ID NO: 18 | Amino acid sequence of a heavy chain of the P74294 |
| | BAFF/B7RP1 bispecific molecule |
| SEQ ID NO: 19 | Amino acid sequence of the immunoglobulin light chain of an |
| | IgG anti-huB7RP1 antibody |
| SEQ ID NO: 20 | Amino acid sequence preceding a heavy chain CDR1 |
| SEQ ID NO: 21 | Amino acid sequence preceding a heavy chain CDR2 |
| SEQ ID NO: 22 | Amino acid sequence following heavy chain CDR3 |
| SEQ ID NO: 23 | Amino acid sequence following light chain CDR3 |
| SEQ ID NO: 24 | Linker |
| SEQ ID NO: 25 | Amino acid sequence of the immunoglobulin heavy chain of an |
| | anti-B7RP1 IgG antibody |
| SEQ ID NO: 26 | Amino acid sequence of heavy chain of construct P71617 |
| SEQ ID NO: 27 | Amino acid sequence of light chain of construct P71618 |
| SEQ ID NO: 28 | Amino acid sequence of heavy chain of construct P71620 |
| SEQ ID NO: 29 | Amino acid sequence of the heavy chain of the P71621 |
| | construct |
| SEQ ID NO: 30 | Amino acid sequence of the heavy chain of construct P71622 |
| SEQ ID NO: 31 | Amino acid sequence of the heavy chain of construct P71623 |
| SEQ ID NO: 32 | Amino acid sequence of αBAFF peptibody |
| SEQ ID NO: 33 | Amino acid sequence of human IgG1 Fc region |
| SEQ ID NO: 34 | Amino acid sequence of human IgG2 Fc region |
| SEQ ID NO: 35 | Amino acid sequence of human IgG3 Fc region |
| SEQ ID NO: 36 | Amino acid sequence of human IgG4 Fc region |
| SEQ ID NO: 37 | Amino acid sequence of a linker |
| SEQ ID NO: 38 | Amino acid sequence of a linker |
| SEQ ID NO: 39 | Amino acid sequence of a linker |
| SEQ ID NO: 40 | Amino acid sequence of a linker |
| SEQ ID NO: 41 | Nucleic acid sequence encoding the amino acid sequence of SEQ ID NO: 1 |
| SEQ ID NO: 42 | Nucleic acid sequence encoding the amino acid sequence of SEQ ID NO: 4 |
| SEQ ID NO: 43 | Nucleic acid sequence encoding the amino acid sequence of SEQ ID NO: 5 |
| SEQ ID NO: 44 | Nucleic acid sequence encoding the amino acid sequence of SEQ ID NO: 6 |
| SEQ ID NO: 45 | Nucleic acid sequence encoding the amino acid sequence of SEQ ID NO: 7 |
| SEQ ID NO: 46 | Nucleic acid sequence encoding the amino acid sequence of SEQ ID NO: 8 |
| SEQ ID NO: 47 | Nucleic acid sequence encoding the amino acid sequence of SEQ ID NO: 9 |
| SEQ ID NO: 48 | Nucleic acid sequence encoding the amino acid sequence of SEQ ID NO: 10 |
| SEQ ID NO: 49 | Nucleic acid sequence encoding the amino acid sequence of SEQ ID NO: 11 |
| SEQ ID NO: 50 | Nucleic acid sequence encoding the amino acid sequence of SEQ ID NO: 12 |
| SEQ ID NO: 51 | Nucleic acid sequence encoding the amino acid sequence of SEQ ID NO: 13 |
| SEQ ID NO: 52 | Nucleic acid sequence encoding the amino acid sequence of SEQ ID NO: 14 |
| SEQ ID NO: 53 | Nucleic acid sequence encoding the amino acid sequence of SEQ ID NO: 15 |
| SEQ ID NO: 54 | Nucleic acid sequence encoding the amino acid sequence of SEQ ID NO: 16 |
| SEQ ID NO: 55 | Nucleic acid sequence encoding the amino acid sequence of SEQ ID NO: 17 |
| SEQ ID NO: 56 | Nucleic acid sequence encoding the amino acid sequence of SEQ ID NO: 18 |
| SEQ ID NO: 57 | Nucleic acid sequence encoding the amino acid sequence of SEQ ID NO: 19 |
| SEQ ID NO: 58 | Nucleic acid sequence encoding the amino acid sequence of SEQ ID NO: 24 |
| SEQ ID NO: 59 | Nucleic acid sequence encoding the amino acid sequence of SEQ ID NO: 25 |
| SEQ ID NO: 60 | Nucleic acid sequence encoding the amino acid sequence of SEQ ID NO: 26 |
| SEQ ID NO: 61 | Nucleic acid sequence encoding the amino acid sequence of SEQ ID NO: 27 |
| SEQ ID NO: 62 | Nucleic acid sequence encoding the amino acid sequence of SEQ ID NO: 28 |
| SEQ ID NO: 63 | Nucleic acid sequence encoding the amino acid sequence of SEQ ID NO: 29 |
| SEQ ID NO: 64 | Nucleic acid sequence encoding the amino acid sequence of SEQ ID NO: 30 |
| SEQ ID NO: 65 | Nucleic acid sequence encoding the amino acid sequence of SEQ ID NO: 31 |
| SEQ ID NO: 66 | Nucleic acid sequence encoding the amino acid sequence of SEQ ID NO: 32 |
| SEQ ID NO: 67 | Nucleic acid sequence encoding the amino acid sequence of SEQ ID NO: 33 |
| SEQ ID NO: 68 | Nucleic acid sequence encoding the amino acid sequence of SEQ ID NO: 34 |
| SEQ ID NO: 69 | Nucleic acid sequence encoding the amino acid sequence of SEQ ID NO: 35 |
| SEQ ID NO: 70 | Nucleic acid sequence encoding the amino acid sequence of SEQ ID NO: 36 |
| SEQ ID NO: 71 | Amino acid sequence of a linker |
| SEQ ID NO: 72 | Amino acid sequence of a linker |

### DETAILED DESCRIPTION

Provided herein are bispecific proteins that bind to and inhibit both B cell activating factor (BAFF; also known as BLYS, TALL1, THANK, or TNFSF13B) and B7-related protein 1 (B7RP1; also known as ICOS Ligand, ICOSL, LICOS, B7 Homolog 2, B7H2, and GL50), nucleic acids encoding these bispecific proteins, and methods of making and using these proteins. The bispecific proteins can inhibit both BAFF-mediated B proliferation and B7RP1-mediated T cell proliferation. In another aspect, the bispecific proteins can inhibit B7RP1 binding to T cells. Such a bispecific protein can be an IgG antibody comprising two different immunoglobulin heavy chains and two different immunoglobulin light chains, where one heavy chain/light chain pair binds to BAFF and the other binds to B7RP1. Alternatively, the B7RP1-binding portion of the bispecific protein can comprise an IgG antibody including two identical heavy chains and two identical light chains, and the BAFF-binding portion of the bispecific protein can comprise one or more BAFF-binding peptides, which can be fused to the anti-B7RP1 antibody, optionally via the N-terminus of the immunoglobulin heavy or light chain, the carboxyterminus of the immunoglobulin heavy chain, and/or within the CH2 and/or CH3 region of the immunoglobulin heavy chain.

### Definitions

An “antibody,” as meant herein, is a protein comprising a heavy and/or light chain immunoglobulin variable region.

A “bispecific” protein, as meant herein is a protein that can bind specifically to two different molecules, which, in some embodiments, are proteins. For example, in some embodiments, a bispecific protein can bind to both BAFF and B7RP1.

A patient is receiving “concurrent” treatment with two or more therapeutics when the patient receives the two or more therapeutics during the same general timeframe, optionally at the very same time. For example, if a patient were dosed with one therapeutic daily on an ongoing basis and were also dosed with another therapeutic once a month on an ongoing basis, the patient would be receiving these two drugs concurrently. Similarly, a patient dosed with two different therapeutics, each administered every two weeks, but not on the same day, would be receiving concurrent treatment with the two therapeutics. Further, a patient receiving one therapeutic on an ongoing basis once per week and another therapeutic once per day for only three days would be receiving treatment for a short period of time with these two therapeutics.

As meant herein, an “Fc region” is a dimer consisting of two polypeptide chains joined by one or more disulfide bonds, each chain comprising part or all of a hinge domain plus a CH2 and a CH3 domain. Each of the polypeptide chains is referred to as an “Fc polypeptide chain.” More specifically, the Fc regions contemplated for use with the present invention are IgG Fc regions, which can be mammalian, for example human, IgG1, IgG2, IgG3, or IgG4 Fc regions. Among human IgG1 Fc regions, at least two allelic types are known. The amino acid sequences an Fc polypeptide chain can vary from those of a mammalian Fc polypeptide by no more than 20, 15, 12, 10, 8, 5, or 3 substitutions, insertions, or deletions of a single amino acid relative to a mammalian Fc polypeptide amino acid sequence. Alternatively or in addition, the amino acid sequence of an Fc polypeptide chain can vary from the sequence of a known or naturally occurring Fc polypeptide chain by no more than 10 insertions, deletions, or substitutions of a single amino acid per every 100 amino acids of sequence. In some embodiments, such variations can be “heterodimerizing alterations” that facilitate the formation of heterodimers over homodimers. In referring to particular positions within an Fc polypeptide chain, the EU numbering system (Edelman et al. (1969), Proc. Natl. Acad. Sci. 63:78-85) is used, as illustrated in the alignment of human IgG Fc polypeptide chains in Table 1 below.

TABLE 1
Alignment of amino acid sequences of human IgG Fc regions

| | |
|---|---|
| IgG1 | ----------------------------------------------- |
| IgG2 | ----------------------------------------------- |
| IgG3 | ELKTPLGDTTHTCPRCPEPKSCDTPPPCPRCPEPKSCDTPPPCPRCP |
| IgG4 | ----------------------------------------------- |
| | 225 235 245 255 265 275 |
| | * * * * * * |
| IgG1 | EPKSCDKTHTCPPCPAPELLGGPSVFLFPPKPKDTLMISRTPEVTCVVVDVSHEDPEVKF |
| IgG2 | ERKCCVE---CPPCPAPPVA-GPSVFLFPPKPKDTLMISRTPEVTCVVVDVSHEDPEVQF |
| IgG3 | EPKSCDTPPPCPRCPAPELLGGPSVFLFPPKPKDTLMISRTPEVTCVVVDVSHEDPEVQF |
| IgG4 | ESKYG---PPCPSCPAPEFLGGPSVFLFPPKPKDTLMISRTPEVTCVVVDVSQEDPEVQF |
| | 285 295 305 315 325 335 |
| | * * * * * * |
| IgG1 | NWYVDGVEVHNAKTKPREEQYNSTYRVVSVLTVLHQDWLNGKEYKCKVSNKALPAPIEKT |
| IgG2 | NWYVDGMEVHNAKTKPREEQFNSTFRVVSVLTVVHQDWLNGKEYKCKVSNKGLPAPIEKT |
| IgG3 | KWYVDGVEVHNAKTKPREEQYNSTFRVVSVLTVLHQDWLNGKEYKCKVSNKALPAPIEKT |
| IgG4 | NWYVDGVEVHNAKTKPREEQFNSTYRVVSVLTVLHQDWLNGKEYKCKVSNKGLPSSIEKT |
| | 345 355 365 375 385 395 |
| | * * * * * * |
| IgG1 | ISKAKGQPREPQVYTLPPSRDELTKNQVSLTCLVKGFYPSDIAVEWESNGQPENNYKTTP |
| IgG2 | ISKTKGQPREPQVYTLPPSREEMTKNQVSLTCLVKGFYPSDIAVEWESNGQPENNYKTTP |
| IgG3 | ISKTKGQPREPQVYTLPPSREEMTKNQVSLTCLVKGFYPSDIAVEWESSGQPENNYNTTP |
| IgG4 | ISKAKGQPREPQVYTLPPSQEEMTKNQVSLTCLVKGFYPSDIAVEWESNGQPENNYKTTP |
| | 405 415 425 435 445 |
| | * * * * * |
| IgG1 | PVLDSDGSFFLYSKLTVDKSRWQQGNVFSCSVMHEALHNHYTQKSLSLSPGK (SEQ ID NO: 33) |
| IgG2 | PMLDSDGSFFLYSKLTVDKSRWQQGNVFSCSVMHEALHNHYTQKSLSLSPGK (SEQ ID NO: 34) |
| IgG3 | PMLDSDGSFFLYSKLTVDKSRWQQGNIFSCSVMHEALHNRFTQKSLSLSPGK (SEQ ID NO: 35) |
| IgG4 | PVLDSDGSFFLYSRLTVDKSRWQEGNVFSCSVMHEALHNHYTQKSLSLSLGK (SEQ ID NO: 36) |

At some positions, naturally-occurring polymorphisms can occur. For example, the methionine at position 282 in the IgG2 sequence given above is more typically a valine in naturally occurring IgG2 sequences. Similarly, the tyrosine at position 296 in an IgG3 sequence can also be a phenylalanine.

“Heterodimerizing alterations” generally refer to alterations in the CH3 regions two different IgG heavy chains that facilitate the formation of heterodimeric heavy chain dimers, that is, dimerized heavy chains that do not have identical amino acid sequences. Heterodimerizing alterations can be asymmetric, that is, one heavy chain having a certain alteration can pair with another heavy chain having a different alteration. These alterations facilitate heterodimerization and disfavor homodimerization. One example of such paired heterodimerizing alterations are the so-called “knobs and holes” substitutions. See, e.g., U.S. Pat. No. 7,695,936 and US Patent Application Publication 2003/0078385, the portions of which describe such mutations are incorporated herein by reference. As meant herein, heavy chain-heavy chain pair that contains one pair of knobs and holes substitutions, contains one substitution in one heavy chain and another substitution in the other heavy chain. For example, the following knobs and holes substitutions have been found to increase heterodimer formation as compared with that found with unmodified heavy chains: 1) Y407T in one chain and T366Y in the other; 2) Y407A in one chain and T366W in the other; 3) F405A in one chain and T394W in the other; 4) F405W in one chain and T394S in the other; 5) Y407T in one chain and T366Y in the other; 6) T366Y and F405A in one chain and T394W and Y407T in the other; 7) T366W and F405W in one chain and T394S and Y407A in the other; 8) F405W and Y407A in one chain and T366W and T394S in the other; and 9) T366W in one polypeptide of the Fc and T366S, L368A, and Y407V in the other. As meant herein, mutations in an Fc polypeptide are denoted in the following way. The amino acid (using the one letter code) normally present at a given position in the CH3 region using the EU numbering system (which is presented in Edelman et al. (1969), Proc. Natl. Acad. Sci. 63:78-85) is followed by the EU position number, which is followed by the alternate amino acid that is present at that position. For example, Y407T means that the tyrosine normally present at EU position 407 is replaced by a threonine. For the sake of clarity, the EU system of numbering is illustrated in Table 1 below. Alternatively or in addition to such alterations, substitutions creating new disulfide bridges can facilitate heterodimer formation. See, e.g., US Patent Application Publication 2003/0078385, the portions of which describe such mutations are incorporated herein by reference. Such alterations in an IgG1 Fc region include, for example, the following substitutions: Y349C in one Fc-polypeptide chain and S354C in the other; Y349C in one Fc-polypeptide chain and E356C in the other; Y349C in one Fc-polypeptide chain and E357C in the other; L351C in one Fc-polypeptide chain and S354C in the other; T394C in one Fc-polypeptide chain and E397C in the other; or D399C in one Fc-polypeptide chain and K392C in the other. Similarly, substitutions changing the charge of a one or more residue, for example, in the CH3-CH3 interface, can enhance heterodimer formation as explained in WO 2009/089004, the portions of which describe such substitutions are incorporated herein by reference. Such substitutions are referred to herein as “charge pair substitutions,” and an Fc region containing one pair of charge pair substitutions contains one substitution in one heavy chain and a different substitution in the other. General examples of charge pair substitutions include the following: 1) R409D, R409E, K409D, or K409E in one chain plus D399K or D399R in the other; 2) N392D, N392E, K392D, or K392E in one chain plus D399K or D399R in the other; 3) K439D or K439E in one chain plus E356K, E356R, D356K, or D356R in the other; and 4) K370D or K370E in one chain plus E357K or E357R in the other. In addition, the substitutions Q355D, Q355E, R355D, R355E, K360D, or K360R in both chains can stabilize heterodimers when used with other heterodimerizing alterations. Specific charge pair substitutions can be used either alone or with other charge pair substitutions. Specific examples of single pairs of charge pair substitutions and combinations thereof include the following: 1) K409E in one chain plus D399K in the other; 2) K409E in one chain plus D399R in the other; 3) K409D in one chain plus D399K in the other; 4) K409D in one chain plus D399R in the other; 5) K392E in one chain plus D399R in the other; 6) K392E in one chain plus D399K in the other; 7) K392D in one chain plus D399R in the other; 8) K392D in one chain plus D399K in the other; 9) K409D and K360D in one chain plus D399K and E356K in the other; 10) K409D and K370D in one chain plus D399K and E357K in the other; 11) K409D and K392D in one chain plus D399K, E356K, and E357K in the other; 12) K409D and K392D on one chain and D399K on the other; 13) K409D and K392D on one chain plus D399K and E356K on the other; 14) K409D and K392D on one chain plus D399K and D357K on the other; 15) K409D and K370D on one chain plus D399K and D357K on the other; 16) D399K on one chain plus K409D and K360D on the other; and 17) K409D and K439D on one chain plus D399K and E356K on the other. Any of these heterodimerizing alterations can be part of an immunoglobulin IgG heavy chain as described herein.

A “human” antibody or protein, as meant herein, is an antibody or protein encoded by a nucleic acid sequence of human origin. A human antibody or protein can be made in cultured non-human cells or in vivo in a transgenic organism into which a nucleic acid molecule encoding the human antibody or protein has been introduced. Alternatively, a human antibody or protein can be made in cultured human cells or in a human in vivo.

An “IgG antibody,” as meant herein, is an antibody that consists essentially of the immunoglobulin domains present in most naturally-occurring IgG antibodies, i.e., a immunoglobulin heavy chain comprising a heavy chain variable (VH) region, a first heavy chain constant (CH1) region, a hinge region, a second heavy chain constant (CH2) region, and a third heavy chain constant (CH3) region and a light chain comprising a light chain variable (VL) region and a light chain constant (CL) region. Numerous sequences of such immunoglobulin domains are reported throughout the scientific literature, e.g., in SEQUENCES OF IMMUNOLOGICAL INTEREST, Public Health Service, N.I.H., Bethesda, MD, 1991. An IgG antibody, as meant herein, is a tetramer consisting essentially of two heavy chains and two light chains. Naturally-occurring antibodies including only two immunoglobulin heavy chains and no immunoglobulin light chains, such as some found in camels and sharks (see, e.g., Muyldermans et al., 2001, J. Biotechnol. 74:277-302; Desmyter et al., 2001, J. Biol. Chem. 276:26285-90; Streltsov et al. (2005), Protein Science 14:2901-2909), are not “IgG antibodies” as meant herein. An IgG antibody can be human or can be from another species. In addition, an IgG antibody can contain no more than 40, 35, 30, 25, 20, 15, 10, or 5 substitutions, insertions, and/or deletions of a single amino acid relative to the amino acid sequence of the heavy or light chains of a naturally occurring IgG antibody.

An “immunoglobulin heavy chain” refers to a heavy chain of an IgG, IgA, IgM, IgE, or IgD antibody or variants thereof containing not more than 40, 30, 25, 20, 15, 10, or 5 insertions, deletions, or substitutions of a single amino acid relative to an immunoglobulin heavy chain encoded by nucleic acid sequences originating in nature. An “immunoglobulin IgG heavy chain” is limited to heavy chains from IgG antibodies or variants thereof containing not more than 40, 30, 25, 20, 15, 10, or 5 insertions, deletions, or substitutions of a single amino acid relative to the amino acid sequence of an IgG heavy chain encoded by nucleic acid sequences originating in nature. An immunoglobulin heavy chain consists essentially of a number of distinct regions or domains including a VH region, a CH1 region, a hinge region, a CH2 region, and a CH3 region. In some other isotypes, i.e., IgM and IgA, additional regions are included downstream from the CH3 region. Immunoglobulin heavy chains and the regions included therein are generally described in, e.g., Carayannopoulos and Capra, Immunoglobulins: Structure and Function, pp. 283-314 in FUNDAMENTAL IMMUNOLOGY, 3^{rd} Ed, Paul, ed., Raven Press, New York, 1993, which is incorporated herein by reference. In addition, numerous sequences of subregions of immunoglobulin heavy chains are known in the art. See, e.g., Kabat et al., SEQUENCES OF PROTEINS OF IMMUNOLOGICAL INTEREST, Public Health Service N.I.H., Bethesda, MD, 1991. In some cases, a polypeptide chain that includes an immunoglobulin heavy chain plus some non-immunoglobulin sequences will be referred to herein as a “heavy chain.”

An “immunoglobulin light chain,” as meant herein, is a kappa or a lambda chain from a human antibody or an antibody from another species. Also included among immunoglobulin light chains, as meant herein, are proteins with no more than 20, 15, 10, or 5 insertions, deletions, and/or substitutions of a single amino acid relative to an immunoglobulin light chain encoded by nucleic acid sequences of natural origin. Immunoglobulin light chains are generally described in, e.g., Carayannopoulos and Capra, Immunoglobulins: Structure and Function, pp. 283-314 in FUNDAMENTAL IMMUNOLOGY, 3^{rd} Ed, Paul, ed., Raven Press, New York, 1993, which is incorporated herein by reference. A immunoglobulin light chain contains a VL region and a CL region. Numerous sequences of these regions are known in the art. See, e.g., Kabat et al., SEQUENCES OF PROTEINS OF IMMUNOLOGICAL INTEREST, Public Health Service N.I.H., Bethesda, MD, 1991. In some cases, a polypeptide chain that includes an immunoglobulin light chain plus some non-immunoglobulin sequences will be referred to herein as a “light chain.”

An “immunoglobulin variable region,” as meant herein, is a VH or VL region, which can be of human origin or from another species. Immunoglobulin variable regions are generally described in, e.g., Carayannopoulos and Capra, Immunoglobulins: Structure and Function, pp. 283-314 in FUNDAMENTAL IMMUNOLOGY, 3^{rd} Ed, Paul, ed., Raven Press, New York, 1993, which is incorporated herein by reference. Also included among immunoglobulin variable regions, as meant herein, are proteins with no more than 20, 15, 10, or 5 insertions, deletions, and/or substitutions of a single amino acid relative to an immunoglobulin variable region encoded by nucleic acid sequences of natural origin. An immunoglobulin variable region contains three hypervariable regions, known as complementarity determining region 1 (CDR1), complementarity determining region 2 (CDR2), and complementarity determining region 3 (CDR3). These regions form the antigen binding site of an antibody. The CDRs are embedded within the less variable framework regions (FR1-FR4). The order of these subregions within a variable region is as follows: FR1-CDR1-FR2-CDR2-FR3-CDR3-FR4. Numerous sequences of immunoglobulin variable regions are known in the art. See, e.g., Kabat et al., SEQUENCES OF PROTEINS OF IMMUNOLOGICAL INTEREST, Public Health Service N.I.H., Bethesda, MD, 1991.

CDRs can be located in a VH region sequence in the following way. CDR1 starts at approximately residue 31 of the mature VH region and is usually about 5-7 amino acids long, and it is almost always preceded by a Cys-Xxx-Xxx-Xxx-Xxx-Xxx-Xxx-Xxx-Xxx (SEQ ID NO: 20) (where “Xxx” is any amino acid). The residue following the heavy chain CDR1 is almost always a tryptophan, often a Trp-Val, a Trp-Ile, or a Trp-Ala. Fourteen amino acids are almost always between the last residue in CDR1 and the first in CDR2, and CDR2 typically contains 16 to 19 amino acids. CDR2 may be immediately preceded by Leu-Glu-Trp-Ile-Gly (SEQ ID NO: 21) and may be immediately followed by Lys/Arg-Leu/Ile/Val/Phe/Thr/Ala-Thr/Ser/Ile/Ala. Other amino acids may precede or follow CDR2. Thirty two amino acids are almost always between the last residue in CDR2 and the first in CDR3, and CDR3 can be from about 3 to 25 residues long. A Cys-Xxx-Xxx almost always immediately precedes CDR3, and a Trp-Gly-Xxx-Gly (SEQ ID NO: 22) almost always follows CDR3.

Light chain CDRs can be located in a VL region in the following way. CDR1 starts at approximately residue 24 of the mature antibody and is usually about 10 to 17 residues long. It is almost always preceded by a Cys. There are almost always 15 amino acids between the last residue of CDR1 and the first residue of CDR2, and CDR2 is almost always 7 residues long. CDR2 is typically preceded by Ile-Tyr, Val-Tyr, Ile-Lys, or Ile-Phe. There are almost always 32 residues between CDR2 and CDR3, and CDR3 is usually about 7 to 10 amino acids long. CDR3 is almost always preceded by Cys and usually followed by Phe-Gly-Xxx-Gly (SEQ ID NO: 23).

A “linker,” as meant herein, is a peptide that links two polypeptides. A linker can be from 1-80 amino acids in length. In some embodiments, a linker can be 2-40, 3-30, or 3-20 amino acids long. In some embodiments, a linker can be a peptide no more than 14, 13, 12, 11, 10, 9, 8, 7, 6, or 5 amino acids long. In other embodiments, a linker can be 5-25, 5-15, 10-20, or 20-30 amino acids long. In other embodiments, a linker can be about, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids long. In many cases, linkers lack free cysteine residues (i.e. and are therefore not involved in disulfide bonds) and also do not contain N-glycosylation sites (that is, Asn-Xxx-Ser/Thr, where X can be any amino acid except proline).

A “peptibody,” as meant herein, is one or more biologically active peptides fused to an Fc region. Shimamoto et al. (2012), mAbs 4 (5): 586-591, the portions of which explain the structure of a peptibody and how to make it are incorporated herein by reference.

A “peptide,” as meant herein, is a polypeptide that consists of a short amino acid sequence, which may or may not be glycosylated and/or contain modified amino acids. A peptide can be from 2 to 75 amino acids long. In some embodiments, a peptide is 3-60, 3-50, 3-40, 3-30, or 3-20 amino acids long. In other embodiments, a peptide can be 5-25, 5-15, 10-20, or 20-30 amino acids long. In other embodiments, a peptide can be about, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids long.

A “therapeutically effective amount” of a drug used to treat a disease is an amount that can reduce the severity of a disease, reduce the severity of one or more symptoms associated with the disease or its treatment, or delay the onset of more serious symptoms or a more serious disease that can occur with some frequency following the treated condition.

“Treatment” of any disease mentioned herein encompasses an alleviation of at least one symptom of the disease, a reduction in the severity of the disease, or the delay or prevention of disease progression to more serious symptoms that may, in some cases, accompany the disease or lead to at least one other disease. Treatment need not mean that the disease is totally cured. A useful therapeutic agent needs only to reduce the severity of a disease, reduce the severity of one or more symptoms associated with the disease or its treatment, or delay the onset of more serious symptoms or a more serious disease that can occur with some frequency following the treated condition. For example, if the disease were an inflammatory bowel disease, a therapeutic agent used as a treatment may reduce the number of distinct sites of inflammation in the gut or the total extent of the gut affected. It may reduce pain and/or swelling, reduce symptoms such as diarrhea, constipation, or vomiting, and/or prevent perforation of the gut. A patient's condition can be assessed by standard techniques such as an x-ray performed following a barium enema or enteroclysis, endoscopy, colonoscopy, and/or a biopsy. Suitable procedures vary according to the patient's condition and symptoms. Similarly, if the disease treated were systemic lupus erythematosus (SLE), disease activity could be evaluated using the SLEDAI index for scoring, as explained below.

### Bispecific Proteins that Bind to BAFF and B7RP1

Disclosed herein are bispecific proteins that bind to B7RP1 and BAFF and/or that can inhibit B7RP1-mediated T cell proliferation and BAFF-mediated B cell proliferation in vitro. The BAFF and B7RP1 proteins to which a bispecific protein as described herein binds can be human proteins and/or can be proteins from another species such as cynomolgus monkey, rhesus monkey, chimpanzee, mouse, and/or rabbit, among others. In some embodiments, a bispecific protein as described herein can, for example, bind to both human (Homo sapiens) and cynomolgus monkey (Macaca fascicularis) B7RP1 and BAFF proteins.

In some embodiments, these bispecific proteins can be bispecific IgG antibodies in which the B7RP1-binding portion and the BAFF-binding portion each consists essentially of an immunoglobulin IgG heavy chain and an immunoglobulin light chain. Thus, such a bispecific antibody contains two different immunoglobulin heavy chains and two different immunoglobulin light chains. Together, these two pairs of immunoglobulin heavy and light chains form a complete bispecific IgG antibody. Bispecific IgG antibodies are known in the art, and a number of other formats for bispecific antibodies are also known. See, e.g., Kontermann, Bispecific Antibodies: Developments and Current Perspectives, pp. 1-28 in BISPECIFIC ANTIBODIES, Kontermann, ed., Springer-Verlag, Berlin, Heidelburg, 2011, the portions of which describe these antibodies are incorporated herein by reference. Antibodies that can bind to BAFF and B7RP1, regardless of format, are contemplated herein. Bispecific IgG antibodies can be human, humanized, or chimeric and can be of the IgG1, IgG2, IgG3, or IgG4 isotype. In some embodiments, bispecific IgG antibodies can be conjugated to other moieties. Amino acid sequences of anti-BAFF and anti-B7RP1 antibodies are known in the art. See e.g., U.S. Pat. No. 7,737,111 and U.S. Patent Application Publication US 2011/0117093. The portions of these documents that describe such antibodies are incorporated herein by reference. In some embodiments, such bispecific antibodies can comprise “heterodimerizing alterations,” as defined above, including charge pair substitutions, that facilitate formation of a heterotetrameric bispecific IgG antibody.

In other embodiments, the bispecific proteins described herein can be fusion proteins comprising an antibody that binds to B7RP1, which comprises an immunoglobulin IgG heavy chain and an immunoglobulin light chain, and a peptide that binds to BAFF. The BAFF-binding peptide can be present in one or multiple copies, such as two, three, four, five, six, seven, eight, or up to 16 copies. The BAFF-binding peptide may bind to BAFF proteins from species such as mouse, cynomolgus monkey, and/or humans, among many other possible species. The antibody can be an anti-B7RP1 IgG antibody, optionally a human or humanized antibody that binds to human and/or cynomolgus monkey B7RP1. In some embodiments, a linker can be attached to the C terminus of the heavy chain of the anti-B7RP1 IgG antibody, followed by a first BAFF-binding peptide, another linker, and a second BAFF-binding peptide. A third, fourth, fifth, sixth, seventh, eighth, or up to sixteenth BAFF-binding peptide can follow these two, optionally interspersed with linkers. Alternatively or in addition, one, two, three, four, five, six, seven, or eight BAFF-binding peptides can be inserted elsewhere in the anti-B7RP1 antibody, for example at the N terminus of the immunoglobulin heavy chain or immunoglobulin light chain or in a loop region in the CH2 or CH3 region. The IgG antibody can be a mammalian antibody, such as a human or murine antibody. The anti-B7RP1 antibody can be a human or humanized IgG1, IgG2, IgG3, or IgG4 antibody. In such bispecific fusion proteins comprising an anti-B7RP1 IgG antibody, the bispecific protein can comprise a heavy chain comprising the amino acid sequence of SEQ ID NO:17 or SEQ ID NO:18 and an immunoglobulin light chain comprising the amino acid sequence of SEQ ID NO:19. Variants comprising a heavy chain having an amino acid sequence containing no more than 30, 25, 20, 15, 10, 5, or 3 insertions, deletions, or substitutions of a single amino acid relative to SEQ ID NO: 17 or 18 are contemplated. Similarly, variants comprising an immunoglobulin light chain having an amino acid sequence containing no more 20, 15, 10, 8, 7, 5, or 3 insertions, deletions, or substitutions or a single amino acid relative SEQ ID NO:19 are contemplated. Such bispecific proteins can be tetramers comprising two polypeptides comprising the amino acid sequence of SEQ ID NO:17 or 18 or a variant thereof and two light chains comprising the amino acid sequence of SEQ ID NO:19 or a variant thereof.

A BAFF-binding peptide portion of a bispecific fusion protein as described above can comprise the amino acid sequence of SEQ ID NO:1, SEQ ID NO:2, or SEQ ID NO: 3. Such BAFF-binding peptides are described in U.S. Pat. No. 7,737,111, the relevant portions of which are incorporated herein by reference. In some embodiments, there may be one, two, three, four, five, six, seven, eight, nine, ten, eleven, twelve, thirteen, fourteen, fifteen, or sixteen copies of such a BAFF-binding peptide present in the bispecific protein. A BAFF-binding peptide can be attached to the carboxy end of the anti-B7RP1 antibody, for example, via a linker. For example, the carboxy end of an anti-B7RP1 IgG antibody can be followed by a linker having, for example, the amino acid sequence of Gly-Gly-Gly-Gly (SEQ ID NO:4). Examples of other suitable linkers include Gly-Gly, Gly-Gly-Gly, Gly-Gly-Gly-Ser (SEQ ID NO:37), Gly-Gly-Gly-Pro (SEQ ID NO:38), Gly-Gly-Gly-Gln (SEQ ID NO:39), and Gly-Gly-Gly-Gly-Gly (SEQ ID NO:40), among many others. This linker can be followed by a BAFF-binding peptide. The BAFF-binding peptide can be followed by another linker comprising, for example, the amino acid sequence of SEQ ID NO:5, SEQ ID NO:6, SEQ ID NO: 7, or SEQ ID NO:24. Other linker could also be used. This linker can be followed by another BAFF-binding peptide comprising, for example, the amino acid sequence of SEQ ID NO:1.

In the bispecific fusion proteins described immediately above or in the bispecific heterotetrameric IgG antibodies described above, a VL region can contain a CDR1, a CDR2, and a CDR3 comprising the amino acid sequences of SEQ ID NO:8, SEQ ID NO:9, and SEQ ID NO:10, respectively. A VH region CDR1, CDR2, and CDR3 can comprise the amino acid sequences of SEQ ID NO:11, SEQ ID NO:12, and SEQ ID NO: 13, respectively. In some embodiments, a VL region of the IgG antibody can comprise the amino acid sequence of SEQ ID NO:14 or a variant thereof, and the VH region can comprise the amino acid sequence of SEQ ID NO:15 or a variant thereof. Such variant sequences can comprise not more than 10 deletions, insertions of substitutions of a single amino acid per 100 amino acids relative to a reference sequence.

### Proteins Comprising a Linker

Provided herein are linkers having the amino acid sequences of SEQ ID NO:5, 6, or 7 that confer favorable physical properties on a protein that contains them. As shown in Example 1 below, the use of two particular linkers, i.e., those having the amino acid sequences of SEQ ID NO:6 and SEQ ID NO:7, had positive effects on properties such as expression, stability, and viscosity of a bispecific molecule. Thus, a variety of proteins containing these linkers may have such favorable properties as compared to similar proteins containing other linkers.

### Therapeutic Uses of Bispecific Proteins

The bispecific proteins binding to BAFF and B7RP1 described herein can be used as therapeutics for a variety of indications, particularly conditions driven by auto-antibodies and/or conditions mediated by both T cells and B cells. Such conditions include, for example, SLE, lupus, nephritis, ANCA-positive vasculitis, rheumatoid arthritis (RA), dermatomyositis, polymyositis, gastrointestinal diseases such as Crohn's disease, ulcerative colitis, and celiac disease, skin conditions such as pemphigus, pemphigoid, and subacute cutaneous lupus erythematosus (SCLE), diseases of the nervous system such as multiple sclerosis and chronic inflammatory demyelinating polyneuropathy (CIDP), neuromuscular diseases such as myasthenia gravis, diseases involving the kidneys such as Goodpasture's syndrome and glomerulonephritis, hematologic conditions such as autoimmune hemolytic anemia (AIHA), idiopathic thrombocytopenia purpura (ITP), and autoimmune neutropenia, liver conditions such as chronic active hepatitis and primary biliary cirrhosis, Sjogren's syndrome, systemic sclerosis, and endocrine conditions including Hashimoto's thyroiditis, Graves' disease, Addison's disease, and multiple endocrine autoimmune failure (commonly including diabetes, hypothyroidism, Addison's disease, and gonadal failure). A therapeutically effective amount of a bispecific protein as described herein can be administered to a patient suffering from any of these conditions to treat the condition.

In one embodiment, a bispecific protein that can inhibit BAFF-mediated B cell proliferation and B7RP1-mediated T cell proliferation can be used to treat a patient suffering from SLE. SLE is an autoimmune disease of unknown etiology marked by autoreactivity to nuclear self antigens. Its clinical manifestations are so diverse that it is questionable whether it is truly a single disease or a group of related conditions. Kotzin (1996) Systemic lupus erythematosus. Cell 85:303-306; Rahman and Isenberg (2008), Systemic lupus erythematosus. N. Engl. J. Med. 358:929-939. Symptoms can include the following: constitutional symptoms such as malaise, fatigue, fevers, anorexia, and weight loss; diverse skin symptoms including acute, transient facial rashes in adults, bullous disease, and chronic and disfiguring rashes of the head and neck; arthritis; muscle pain and/or weakness; cardiovascular symptoms such as mitral valve thickening, vegetations, regurgitation, stenosis, pericarditis, and ischemic heart disease, some of which can culminate in stroke, embolic disease, heart failure, infectious endocarditis, or valve failure; nephritis, which is a major cause of morbidity in SLE; neurological symptoms including cognitive dysfunction, depression, psychosis, coma, seizure disorders, migraine, and other headache syndromes, aseptic meningitis, chorea, stroke, and cranial neuropathies; hematologic symptoms including leucopenia, thrombocytopenia, serositis, anemia, coagulation abnormalities, splenomegaly, and lymphadenopathy; and various gastrointestinal abnormalities. Id; Vratsanos et al., “Systemic Lupus Erythematosus,” Chapter 39 in Samter's Immunological Diseases, 6^{th} Edition, Austen et al., eds., Lippincott Williams & Wilkins, Philadelphia, PA, 2001. Severity of symptoms varies widely, as does the course of the disease. SLE can be deadly.

An SLE patient can be treated with a bispecific protein that inhibits BAFF and B7RP1 before, after, or concurrently with treatment using an existing therapy for SLE. Such existing therapies for SLE include corticosteroids such as prednisone, prednisolone, and methylprednisolone, antimalarials such as hydroxychloroquine, quinacrine, and chloroquine, retinoic acid, aspirin and other nonsteroidal anti-inflammatory drugs (NSAIDs), cyclophosphamide, dehydroepiandrosterone, mycophenolate mofetil, azathioprine, chlorambucil, methotrexate, tacrolimus, dapsone, thalidomide, leflunomide, cyclosporine, belimumab, anti-CD20 antibodies such as rituximab, and fusion proteins such as abatacept.

The disease activity of SLE patients can be rated using an instrument such as the Systemic Lupus Erythrematosus Disease Activity Index (SLEDAI), which provides a score for disease activity that takes into consideration the following symptoms, which are weighted according to severity: seizure, psychosis, organic brain syndrome, visual disturbance, cranial nerve disorder, lupus headache, vasculitis, arthritis, myositis, urinary casts, hematuria, proteinuria, pyuria, new rash, alopecia, mucosal ulcers, pleurisy, pericarditis, low complement, increased DNA binding, fever, thrombocytopenia, and leucopenia. Bombardier et al. (1992), Arthr. & Rheum. 35 (6): 630-640, the relevant portions of which are incorporated herein by reference. The treatments described herein can be useful in lessening or eliminating symptoms of SLE as measured by SLEDAI. Methods of treatment described herein can improve a patient's SLEDAI score compared to a baseline value for the same patient prior to initiation of treatment with a bispecific protein as described herein.

Another method for assessing disease activity in SLE is the British Isles Lupus Assessment Group (BILAG) index, which is a disease activity assessment system for SLE patients based on the principle of the physician's intention to treat. Stoll et al. (1996), Ann. Rheum Dis. 55:756-760; Hay et al. (1993), Q. J. Med. 86:447-458. The portions of these references describing the BILAG are incorporated herein by reference. A BILAG score is assigned by giving separate numeric or alphabetic disease activity scores in each of eight organ-based systems, general (such as fever and fatigue), mucocutaneous (such as rash and alopecia, among many other symptoms), neurological (such as seizures, migraine headaches, and psychosis, among many other symptoms), musculoskeletal (such as arthritis), cardiorespiratory (such as cardiac failure and decreased pulmonary function), vasculitis and thrombosis, renal (such as nephritis), and hematological. Id. The treatments described herein can be useful in lessening or eliminating symptoms of SLE as measured by the BILAG index or in decreasing a patient's BILAG score as compared to a baseline value prior to the initiation of treatment with a bispecific protein as described herein.

A bispecific protein as described herein, which inhibits BAFF-mediated proliferation of B cells and B7RP1-mediated proliferation of T cells, could also be used to treat rheumatoid arthritis (RA). RA is a chronic disease with systemic symptoms, as well as symptoms relating specifically to the joints. Symptoms commonly include synovitis, leading to painful and swollen joints, and various laboratory abnormalities such as higher-than-normal levels of rheumatoid factor, anti-citrulline modified protein (anti-CCP) antibodies, and C-reactive protein (CRP) and an elevated erythrocyte sedimentation rate (ESR). Less common symptoms include various extra-articular symptoms involving, e.g., tendons, ligaments, blood vessels, the heart, and the lungs. Disease activity can be often measured using a variety of indices. See, e.g., Anderson et al. (2012), Arthritis care & Res. 64 (5): 640-647, the portions of which discuss such indices are incorporated herein by reference. Elements included in such scoring indices include the number of tender joints, the number of swollen joints, functional assessments, and various laboratory findings such as CRP, ESR, etc.

In some embodiments, a patient suffering from RA can be treated with a bispecific protein that inhibits BAFF-mediated B cell proliferation and B7RP1-mediated T cell proliferation before, after, or concurrently with treatment with a drug in current use for RA. Therapeutics currently in use for rheumatoid arthritis (RA) include non-steroidal anti-inflammatory drugs (NSAIDs) (such aspirin and cyclooxygenase-2 (COX-2) inhibitors), disease modifying anti-inflammatory drugs (DMARDs, such as methotrexate, leflunomide, and sulfasalazine), anti-malarials (such as hydroxychloroquine), cyclophosphamide, D-penicillamine, azathioprine, gold salts, tumor necrosis factor inhibitors (such as etanercept, infliximab, adalimumab, golimumab, and certolizumab pegol), CD20 inhibitors such as rituximab, IL-1 antagonists such as anakinra, IL-6 inhibitors such as tocilizumab, inhibitors of Janus kinases (JAKs, such as tofacitinib), abatacept, and corticosteroids, among others.

A therapeutically effective amount of a bispecific protein as described herein, which inhibits BAFF-mediated proliferation of B cells and B7RP1-mediated proliferation of T cells, can also be used to treat an inflammatory bowel disease, such as Crohn's disease or ulcerative colitis. Crohn's disease involves an abnormal inflammation of any portion of the alimentary tract from the mouth to the anus, although in most patients abnormal inflammation is confined to the ileocolic, small-intestinal, and colonic-anorectal regions. Typically, the inflammation is discontinuous. Common symptoms include abdominal pain, anorexia, weight loss, fever, diarrhea, fullness and/or tenderness in the right lower quadrant of the abdomen, constipation, vomiting, and perianal discomfort and discharge. Other possible symptoms include peripheral arthritis, growth retardation, episcleritis, aphthous stomatitis, erythema nodosum, pyoderma gangrenosum, kidney stones, impaired urinary dilution and alkalinization, malabsorption, and gallstones, among others. See e.g. Strober et al., Medical Immunology, 10^{th} Edition, Section III, Ch. 35 (2001); Merck Manual of Diagnosis and Therapy, 17^{th} Edition, Section 3, Ch. 31 (1999). Macrophages isolated from patients with Crohn's disease produce increased amounts of IL-12, IFNγ, TNFα, and other inflammatory cytokines.

Ulcerative colitis, though it is sometimes hard to distinguish from Crohn's disease, is distinct from Crohn's disease in several respects. First, it is generally limited to the colon while Crohn's disease may occur throughout the alimentary tract. Second, ulcerative colitis mainly involves inflammation only of the superficial layers of the bowel, unlike Crohn's disease in which the inflammation can penetrate all way through the wall of the bowel or other location in the alimentary tract. Finally, ulcerative colitis typically involves a continuous area of inflammation, rather than the discontinuous sites of inflammation typical of Crohn's disease. Like Crohn's disease, ulcerative colitis is found primarily in urban areas. Also, genetic factors likely play a role in ulcerative colitis since there is a familial aggregation of cases. Autoantibodies are observed in ulcerative colitis patients more often than Crohn's disease patients. The autoantibodies are often directed to colonic epithelial cell components. Among the most common are antineutrophil cytoplasmic antibodies with specificities for catalase, α-enolase, and lactoferrin. In some cases such antibodies cross react with colonic microorganisms.

In clinical trials, Crohn's disease activity is often scored using the Crohn's Disease Activity Index (CDAI). The CDAI provides a disease activity score based on eight factors including (1) the number of liquid or soft stools per day, (2) a patient rating of the amount of abdominal pain per day, (3) a patient rating of general well-being, (4) a patient report of other symptoms including arthritis, iritis, uveitis, erythema nodosum, pyoderma gangrenosum, ephthous stomatitis, anal fissure, fitula, or abscess, other fistula, or fever, (5) patient report of taking lomotil or other opiates for diarrhea, (6) abdominal mass, (7) hematocrit, and (8) body weight. See, e.g., Best et al. (1976), Gastroenterol. 70:439-444, the relevant portions of which are incorporated herein by reference.

Symptoms of ulcerative colitis are variable. They may include diarrhea, tenesmus, abdominal cramps, blood and mucus in the stool, fever, and rectal bleeding. Toxic megacolon, a potentially life-threatening condition in which the colon is dilated beyond about 6 centimeters and may lose its muscular tone and/or perforate, may also occur. Other symptoms that may accompany ulcerative colitis include peripheral arthritis, ankylosing spondylitis, sacroiliitis, anterior uveitis, erythema nodosum, pyoderma gangrenosum, episcleritis, autoimmune hepatitis, primary sclerosing cholangitis, cirrhosis, and retarded growth and development in children.

In some embodiments a patient suffering from an inflammatory bowel disease (IBD), such as Crohn's disease or ulcerative colitis, can be treated with a bispecific protein that binds to BAFF and B7RP1 before, after, or concurrently with treatment with an existing therapy for IBD. Existing therapeutics for IBD include, for example, sulfasalazine, 5-aminosalicylic acid and its derivatives (such as olsalazine, balsalazide, and mesalamine), anti-TNF antibodies (including infliximab, adalimumab, golimumab, and certolizumab pegol), corticosteroids for oral or parenteral administration (including prednisone, methylprednisone, budesonide, or hydrocortisone), adrenocorticotropic hormone, antibiotics (including metronidazole, ciprofloxacin, or rifaximin), azathioprine, 6-mercaptopurine, methotrexate, cyclosporine, tacrolimus, and thalidomide.

### Nucleic Acids Encoding Bispecific Proteins

Provided herein are nucleic acids encoding a bispecific protein that can inhibit B7RP1-mediated T cell proliferation and BAFF-mediated B cell proliferation. For example, SEQ ID NO:52 encodes the VL region having the amino acid sequence of SEQ ID NO:14, and SEQ ID NO:53 encodes the VH region having the amino acid sequence of SEQ ID NO:15. Similarly, SEQ ID NOs: 55 and 56 encode the amino acid sequences of SEQ ID NOs: 17 and 18, respectively, which are polypeptides comprising the heavy chain of an anti-B7RP1 antibody fused to two BAFF-binding peptides. SEQ ID NO: 57 encodes the light chain of an anti-B7RP1 antibody, which can be part of a hetero-tetrameric bispecific IgG antibody or a bispecific fusion protein, as described above. Any nucleic acid sequence encoding any amino acid sequence provided herein is contemplated. Similarly, nucleotide sequence variants including silent mutations relative to sequences disclosed herein or encoding the amino acid sequence variants described above are also included within the ambit of the invention. More specifically, nucleotide sequences encoding amino acid sequences that vary by no more than 10 insertions, deletions, or substitutions of a single amino acid per 100 amino acids from amino acid sequences disclosed herein are contemplated.

Nucleic acid sequences encoding bispecific proteins described herein can be determined by one of skill in the art based on the amino acid sequences provided herein and knowledge in the art. Besides more traditional methods of producing cloned DNA segments encoding a particular amino acid sequence, companies such as DNA 2.0 (Menlo Park, CA, USA) and BlueHeron (Bothell, WA, USA), among others, now routinely produce chemically synthesized, gene-sized DNAs of any desired sequence to order, thus streamlining the process of producing such DNAs. Codon usage can be adjusted so as to optimize expression in the system of choice.

### Methods of Making Bispecific Proteins that Bind to BAFF and B7RP1

Nucleic acids encoding the bispecific proteins described herein can be inserted into vectors appropriate for the host cell in which the nucleic acid will be expressed. These nucleic acids can be introduced into the host cells by any of the methods well-known in the art. Host cells that can be used include bacteria, including Escherichia coli, yeast, including Saccharomyces cerevisiae or Pichia pastoris, insect cells including Spodoptera frugiperda cells, plant cells, and mammalian cells, including Chinese hamster ovary (CHO) cells, baby hamster kidney (BHK) cells, monkey kidney cells, Hela cells, human hepatocellular carcinoma cells, and 293 cells, among many others. These host cells can be cultured under conditions such that the introduced nucleic acids will be expressed, and the bispecific protein can be recovered from the culture supernatant or the cell mass.

Generally, the procedure used to introduce the nucleic acids into the host cells may depend upon the host cell into which the nucleic acids are to be introduced. Methods of introducing nucleic acids into bacteria are well-known in the art. For example, electroporation or calcium chloride transformation are commonly used. Methods for introduction of nucleic acids into yeast are also well-known in the art and include, for example, transformation methods using lithium acetate and polyethylene glycol. Methods for introducing heterologous polynucleotides into mammalian cells are well known in the art and include, but are not limited to, dextran-mediated transfection, calcium phosphate precipitation, polybrene mediated transfection, protoplast fusion, electroporation, encapsulation of the polynucleotide(s) in liposomes, and direct microinjection of the DNA into nuclei.

Expression vectors used in any of the host cells can contain sequences necessary for DNA replication, selection of host cells containing the vector, and expression of the exogenous nucleotide sequences. Such sequences can typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Numerous expression vectors appropriate for expression in various host cells are known in the art and are commercially available.

### Pharmaceutical Compositions, Dosing, and Methods of Administration

Pharmaceutical compositions comprising the bispecific proteins described herein are provided. Such compositions can comprise a therapeutically effective amount of a bispecific protein with one or more additional components such as a physiologically acceptable carrier, excipient, or diluent. Such additional components can include buffers, carbohydrates, polyols, amino acids, chelating agents, stabilizers, and/or preservatives, among many possibilities. Many such additional components are described in, e.g., REMINGTON'S PHARMACEUTICAL SCIENCES, 18th Edition, (A. R. Gennaro, ed.), 1990, Mack Publishing Company, the relevant portions of which are incorporated herein by reference.

Dosing of the bispecific proteins described herein can be adjusted to achieve the desired effects. In many cases, repeated dosing will be required because of the chronic nature of the disease being treated. For example, a bispecific protein as described herein can be dosed twice per week, once per week, once every two, three, four, five, six, seven, eight, nine, or ten weeks, or once every two, three, four, five, or six months. The amount of the bispecific protein administered on each day that it is administered can be from about 0.0036 mg to about 700 mg. Alternatively, the dose can calibrated according to the estimated skin surface of a patient, and each dose can be from about 0.002 μg/m² to about 350 mg/m². In another alternative, the dose can be calibrated according to a patient's weight, and each dose can be from about 0.000051 mg/kg to about 10.0 mg/kg.

The bispecific proteins, or pharmaceutical compositions containing these molecules, can be administered by any feasible method. Therapeutics that comprise a protein will ordinarily be administered by a parenteral route, for example by injection, since oral administration, in the absence of some special formulation or circumstance, would lead to hydrolysis of the protein in the acid environment of the stomach. Subcutaneous, intramuscular, intravenous, intraarterial, intralesional, and peritoneal bolus injections are possible routes of administration. The bispecific proteins can also be administered via infusion, for example intravenous or subcutaneous infusion. Topical administration is also possible, especially for diseases involving the skin. Alternatively, the bispecific proteins can be administered through contact with a mucus membrane, for example by intra-nasal, sublingual, vaginal, or rectal administration or administration as an inhalant. Alternatively, certain appropriate pharmaceutical compositions comprising a bispecific protein can be administered orally.

Having described the invention in general terms above, the following examples are offered by way of illustration and not limitation.

### EXAMPLES

### Example 1: Designing and Testing a BAFF/B7RP1 Bispecific Molecule for Human Therapeutic Use

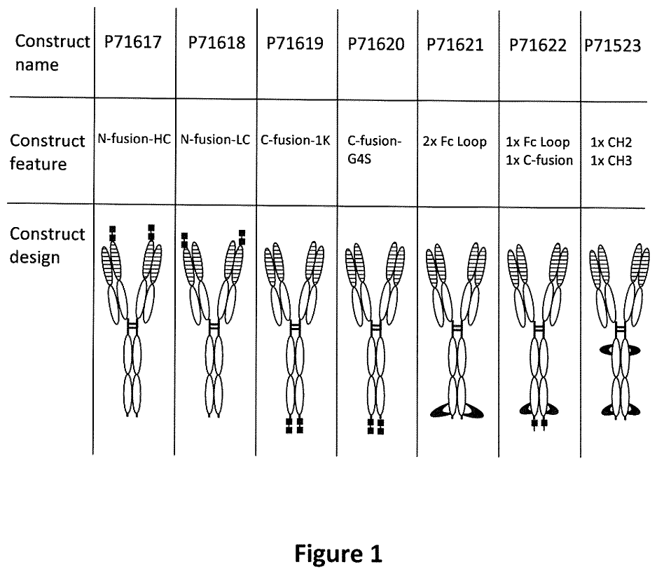

The object of this series of experiments was to find a bispecific molecule that (1) inhibits BAFF-mediated B cell proliferation and B7RP1-mediated T cell proliferation, (2) is highly active in biological assays, and (3) has favorable biophysical properties. A number of schematic designs for the fusion of a peptide that binds human BAFF to an anti-human B7RP1 IgG antibody (anti-huB7RP1) are illustrated in FIG. 1. The sequence of the BAFF-binding peptide is provided in SEQ ID NO:1, and the sequences of the immunoglobulin heavy and light chains of anti-huB7RP1 are provided in SEQ ID NO:25 and SEQ ID NO:19, respectively.

To determine which design had the best biophysical properties, while retaining biological activity, the bispecific molecules diagrammed in FIG. 1 were made and tested. In one construct, two tandem copies of the BAFF-binding peptide with an intervening linker (the “1K linker,” having the amino acid sequence of SEQ ID NO: 24) were fused to the N-terminus of either the immunoglobulin heavy chain (P71617) or immunoglobulin light chain (P71618) of anti-huB7RP1. See FIG. 1. The amino acid sequence of the P71617 heavy chain is provided in SEQ ID NO:26, and the amino acid sequence of the light chain of P71617 is the same as that of the immunoglobulin light chain of anti-huB7RP1 (SEQ ID NO:19). The amino acid sequence of the P71618 light chain is provided in SEQ ID NO:27, and the amino acid sequence of the heavy chain of P71618 is the same as the immunoglobulin heavy chain of anti-huB7RP1 (SEQ ID NO:25). Two tandem copies of the BAFF-binding peptide were also fused to the C-terminal end of the immunoglobulin heavy chain of anti-huB7RP1 (having the amino acid sequence of SEQ ID NO:25) using either the 1K linker mentioned above (having the amino acid sequence of SEQ ID NO:24; P71619) or a 5X(G4S) linker (SEQ ID NO: 71) between the two BAFF-binding peptides (P71620). The amino acid sequences of the heavy chains of these two fusion constructs are provided in SEQ ID NO:16 (P71619) and SEQ ID NO:28 (P71620). In construct P71621, two tandem copies of the BAFF-binding peptide with an intervening 1K linker were inserted into the antibody's CH3 domain between residues 358 and 359 of the amino acid sequence of SEQ ID NO:25 (the amino acid sequence of the immunoglobulin heavy chain of the anti-huB7RP1 antibody). The sequence of the heavy chain of the P71621 construct is provided in SEQ ID NO:29. In construct P71622, the BAFF-binding peptide was inserted into the CH3 domain of the immunoglobulin heavy chain of anti-huB7RP1 (between residues 358 and 359 of SEQ ID NO:25 and a second copy of the BAFF-binding peptide was fused to the C-terminal end of the heavy chain. The amino acid sequence of the heavy chain of P71622 is provided in SEQ ID NO:30. In construct P71623, one BAFF-binding peptide was inserted into the CH2 region (between residues 268 and 269 of SEQ ID NO:25), and a second BAFF-binding peptide was inserted into the CH3 region (between residues 358 and 359 of SEQ ID NO:25). SEQ ID NO:31 is the amino acid sequence of the heavy chain of P71623. Constructs P71619-P71623 all have the immunoglobulin light chain of anti-huB7RP1 (SEQ ID NO:19).

In constructs P74293 and P74294, the linker between the two tandem copies of the BAFF-binding peptides in construct P71619 was modified. The amino acid sequences of the heavy chains of P74293 and P74294 are provided in SEQ ID NO:17 and SEQ ID NO:18, respectively. The immunoglobulin light chains of these constructs also have the amino acid sequence of SEQ ID NO:19.

Nucleic acids encoding the constructs described above were made as follows. Nucleic acids encoding the N-terminal portion of the N-terminal BAFF peptide fusions (P71617 and P71618), including two copies of the BAFF-binding peptide plus an immunoglobulin heavy or light chain variable region, were generated synthetically. These were ligated, through convenient restriction endonuclease sites, to nucleic acids encoding the immunoglobulin heavy or light chain constant region in appropriate vectors. Nucleic acids encoding the heavy chain constant region C-terminal fusions (P71619 and P71620), Fc-loop insertions (P71621 and P71623), and the Fc-loop insertion/C-terminal fusion (P71622) were all generated synthetically and ligated into a vector containing the heavy chain variable region through convenient restriction endonuclease sites.

The various bispecific constructs described above were expressed in both transiently transfected 293 cells and stably transfected CHO cells. The fusion proteins were purified and tested for biological activity. No differences were observed in proteins produced in these two different kinds host cells.

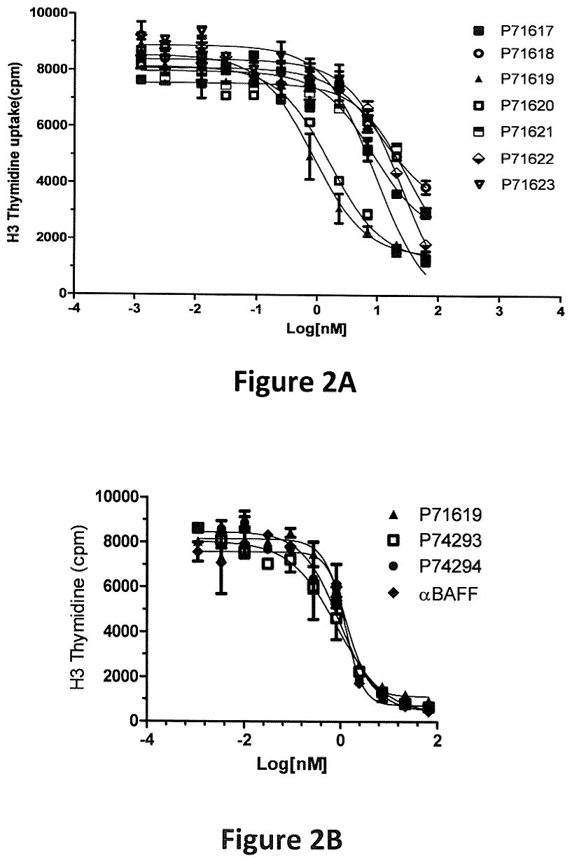

The BAFF inhibitory activities of the bispecific molecules were tested in a BAFF-mediated human primary B cell proliferation assay. In brief, human B cells were purified from peripheral blood mononuclear cells (PBMCs) using negative selection using a human B cell kit II from Miltenyi Biotec (Auburn, CA). About 105 purified B cells were cultured in 96 well microtiter plates in Minimal Essential Media (MEM) plus 10% heat inactivated fetal bovine serum (FBS) in the presence of 50 ng/ml human BAFF protein, 2 μg/ml goat F(ab′) 2 anti-human IgM (Jackson ImmunoResearch), and varying concentrations of one of the bispecific proteins described above at 37° C. in 5% CO² for 48 hours. An anti-BAFF peptibody was used as a positive control (“αBAFF,” which is a homodimer containing two polypeptide chains, each comprising two BAFF-binding peptides fused to an Fc polypeptide). The αBAFF molecule is described in detail in U.S. Pat. No. 7,259,137, and the amino acid sequence of one polypeptide chain of this homodimer is provided in SEQ ID NO:32. The portions of U.S. Pat. No. 7,259,137 describing αBAFF are incorporated herein by reference. Proliferation was measured by the uptake of radioactive 3H-thymidine during the last 18 hours of incubation. Results are shown in FIGS. 2A and 2B.

The data in FIG. 2A indicate that the two C-terminal fusion constructs (P71619 and P71620) were comparable to each other in inhibition of BAFF-mediated B cell proliferation and more potent than all of the other fusion constructs tested in this experiment. P71620 was not pursued further because it tended to aggregate, a property that is highly undesirable in a therapeutic protein. The data in FIG. 2B indicate that P71619 is comparable to the two slightly modified versions of this construct described above (P74293 and P74294) and to a positive control (αBAFF) in inhibition of BAFF-mediated B cell proliferation. Thus, among the bispecific constructs tested, P71619, P71620, P74293, and P74294 had comparable activity in this assay of BAFF-mediated B cell proliferation and better activity than all other constructs tested.

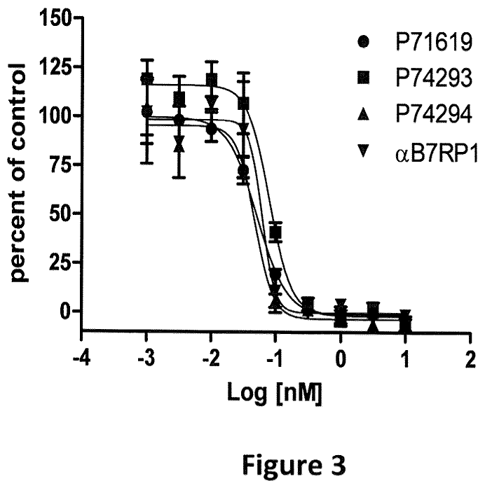

The B7RP1 inhibitory activity of P71619, P74293, and P74294 was assayed using a human B7RP1-Fc-mediated T cell proliferation assay. Primary human T cells purified from PBMCs from healthy human donors using Pan T cell isolation kit from Miltenyi Biotec (Auburn, CA) and stimulated with plate-bound anti-CD3 (1 μg/mL) antibody and a B7RP1-Fc fusion protein (3 μg/mL) in the presence of varying concentrations of the bispecific proteins described above or an IgG2 anti-human B7RP1 antibody (referred to herein as “αB7RP1”). ³H-thymidine was added to the cells after 48 hours, and incorporation of the 3H-thymidine was measured 24 hours later. All of the bispecific antibodies that were tested had similar IC₅₀'s, which were similar to that of αB7RP1 (FIG. 3). Thus, these data suggest that the conjugation of the BAFF-binding peptides to the anti-huB7RP1 antibody had little or no effect on the ability of the antibody to inhibit B7RP1 activity.

The binding affinities of the heterodimeric bispecific antibodies P74293 and P74294 to BAFF and B7RP1 were measured by Kinetic Exclusion Assay (KinExA®; Sapidyne Instruments, Boise, Idaho). Both antibodies have high binding affinities to human BAFF (having K_{d}'s of approximately 30 pM) and to human B7RP1 (having K_{d}'s of approximately 40 pM). See Table 2 below. In addition, both of these bispecifics have similar binding affinities to cynomolgus monkey BAFF compared to human BAFF and to cynomolgus monkey B7RP1 compared to human B7RP1. Table 2.

TABLE 2
binding affinity and cellular potency of P74293 and P74294.

| | P74293 | P74294 |
|---|---|---|
| K_{d} (pM) for binding to human BAFF | 29 | 37 |
| K_{d} (pM) for binding to cynomolgus monkey BAFF | 22.3 | 17.4 |
| IC₅₀ (nM) for inhibition of BAFF-mediated human | 0.86 | 0.96 |
| B cell proliferation | | |
| IC₅₀ (nM) for inhibition of BAFF-mediated | 1.6 | 1.8 |
| cynomolgus monkey B cell proliferation | | |
| K_{d} (pM) for binding to human B7RP1 | 38 | 41 |
| K_{d} (pM) for binding to cynomolgus monkey B7RP1 | 49.4 | 45.2 |
| IC₅₀ (nM) for inhibition of B7RP1-mediated human | 1.36 | 0.98 |
| T cell proliferation | | |
| IC₅₀ (nM) for inhibition of B7RP1-mediated | 0.29 | ND* |
| cynomolgus monkey T cell proliferation | | |

*ND means not determined.

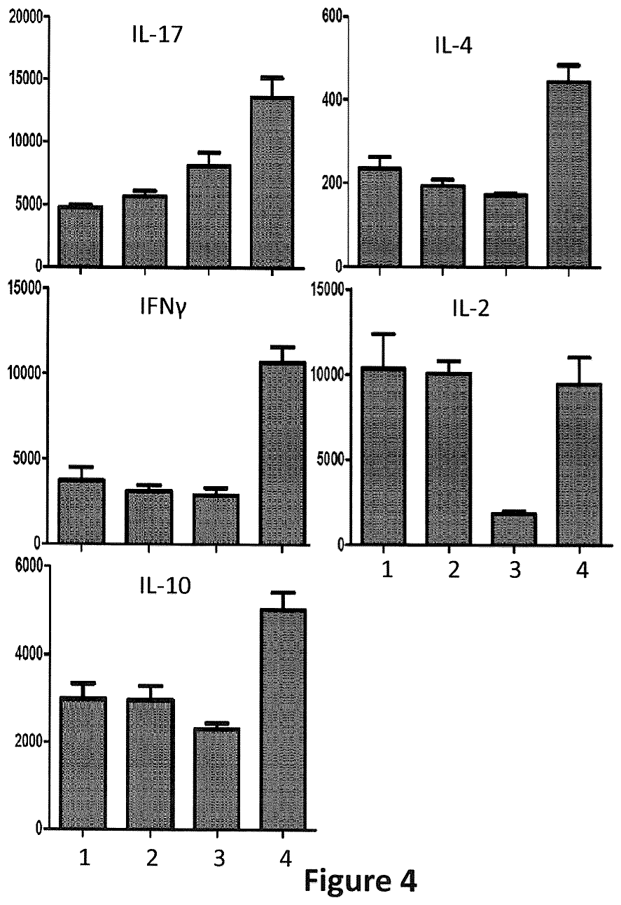

To further assess the activity of P74293 in an in vitro system using human cells, cytokine production by human tonsil cells activated by Staphylococcus enterotoxin B (SEB) was assessed in the presence of various test molecules. Briefly, human tonsil cells were isolated from tissue and stimulated with SEB (1 μg/mL) in the presence of one of the following molecules: (1) αB7RP1, (2) P74293, (3) CTLA4-Ig (a positive control), or (4) human IgG (a negative control). After 72 hours of culture, the cell supernatant was collected, and cytokine levels were assayed using kits from Meso Scale Discovery according to the manufacturer's instructions. Results are shown in FIG. 4.

All three of αB7RP1, P74293, and CTLA4-Ig, bars 1, 2, and 3, respectively in all panels of FIG. 4, inhibited release of IL-17, IL-10, IL-4, and IFNγ. Release of IL-2 was inhibited only by CTLA4-Ig. Thus, αB7RP1 and the anti-BAFF/B7RP1 bispecific P74293 had comparable and specific effects on cytokine secretion by SEB-activated human tonsil cells.

Three heterodimeric bispecific proteins, that is, P71619, P74293, and P74294, were examined for additional properties. Protein titers from cultures of host cells producing these proteins indicated that P74293 and P74294 were produced at about twice the levels at which P71619 was produced. P74293 and P74294 were also more stable than P71619 after storage for two weeks at 40° C. as assessed by size exclusion chromatography (SEC). P74293 formed a clear solution at the onset of storage and after 4 weeks of storage, whereas solutions containing P74394 were hazy at all time points. Solutions of P74293 and P74294 were less viscous than solutions of P71619. Thus, P74293 and P74294 were expressed at higher levels than P71619 and were also more stable and less viscous in the concentration range tested than P71619. The most obvious difference between these molecules lies in the linker between the two BAFF-binding peptides. These data suggest that the linkers in P74293 and P74294 (SEQ ID NOs: 6 and 7) can confer improved properties upon these molecules.

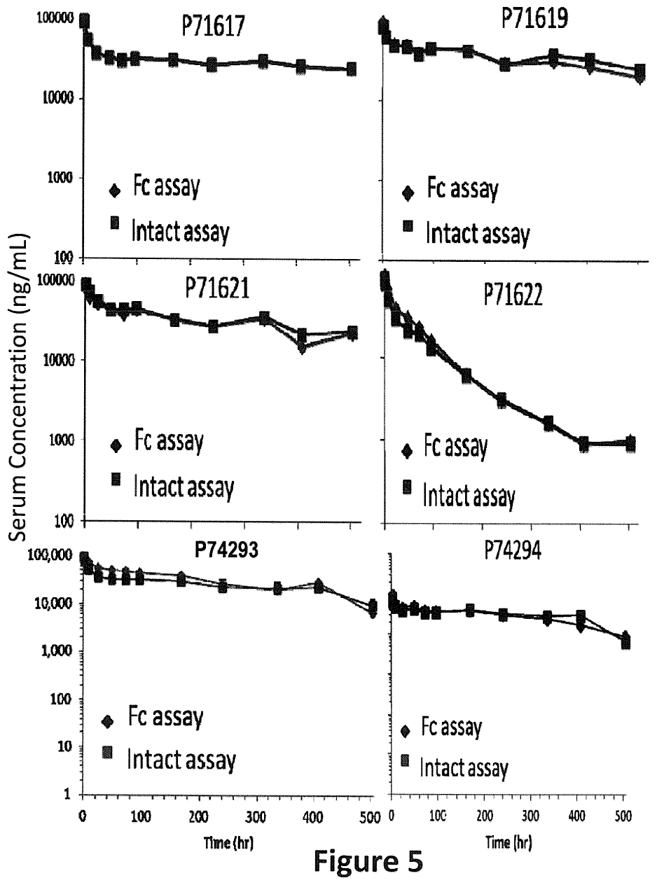

The pharmacokinetic properties of the bispecific molecules described were evaluated in mice. Male CD-1 mice were given a single intravenous (IV) dose (5 mg/kg) of the bispecific fusion proteins P71617, P71619, P71621, P71622, P74293, or P74294. Serum samples were collected before dosing and at 0.5, 2, 8, 24, 48, 72, 96, 168, 240, 336, 408, 504 hours after dosing. The concentration of the bispecific molecule in the serum was determined by two ELISA methods, one registering the presence of the Fc portion and one registering the presence of both the Fc portion and the BAFF-binding peptide portion. For the Fc portion measurement, a biotinylated anti-Fc antibody was used as capture reagent, and ALEXA FLUOR® 647-labeled anti-Fc antibody was used as the detection reagent. To detect the BAFF-binding portion and the Fc portion of the bispecific, a biotinylated BAFF protein was used as the capture reagent, and ALEXA FLUOR® 647-labeled anti-Fc antibody was used as the detection reagent. The bispecific proteins with two tandem copies of BAFF-binding peptides fused to the N-terminus (P71617), C-terminus (P71619, P74293 and P74294) or CH3 domain (P71621) of the heavy chain have very similar PK profiles in mice. FIG. 5. The bispecific protein with one copy of BAFF-binding peptide inserted into the CH3 domain and another copy fused to the C-terminal end of the heavy chain (P71622) had lower exposure compared to the other bispecific proteins. FIG. 5. In addition, the two different ELISA assays resulted in similar serum concentrations of the bispecific proteins, suggesting that no significant cleavage of the bispecific proteins occurred in vivo.

Pharmacokinetic and pharmacodynamic parameters of the P74293 and P74294 heterodimeric bispecific antibodies were also assessed by a single dose study in cynomolgus monkeys. Naïve male cynomolgus monkeys (n=4) were given a single bolus intravenous or subcutaneous dose of P74293 (10 mg/kg), or a single subcutaneous dose of P74294 (10 mg/kg). Both bispecific molecules have PK profiles similar to that of an IgG antibody. The observed pharmacokinetic parameters for P74293 and P74294, as well as for anti-huB7RP1, are reported in Table 3 below.

TABLE 3
Pharmacokinetic parameters in cynomolgus monkey

| | P74293 | | P74294 | Anti-huB7RP1 | |
|---|---|---|---|---|---|
| | 10 | 10 | 10 | 10 | 10 |
| | mg/kg | mg/kg | mg/kg | mg/kg | mg/kg |
| | IV | SC | SC | IV | SC |
| Maximum drug | 323 | 90 | 74 | 264 | 112 |
| concentration (Cₘₐₓ; | | | | | |
| μg/ml) | | | | | |
| Time at which Cₘₐₓ was | | 45 | 51 | | 72 |
| observed (Tₘₐₓ; hr) | | | | | |
| Area under the curve | 33800 | 20300 | 22000 | 26100 | 23800 |
| (AUC_{0-inf}; μg*hr/ml) | | | | | |
| Mean residence time | 136 | 132 | 148 | 138 | 144 |
| (MRT_{0-inf}; hr) | | | | | |
| Total clearance (CL; | 0.303 | 0.491 | 0.484 | 0.388 | 0.427 |
| ml/hr/kg) | | | | | |
| Volume of distribution at | 42.5 | | | 52.1 | |
| steady state (Vss; ml/kg) | | | | | |

The data in Table 3 indicate that the pharmacokinetic parameters of P74293 and P75294 are comparable to each other and to those of anti-huB7RP1 antibody.

### Example 2: Designing and Testing a Murine Bispecific Surrogate Molecule

To conduct preclinical studies in mice, a murine surrogate bispecific molecule that could bind to murine B7RP1 and murine BAFF (hereinafter, the “murine surrogate”) was constructed. The anti-huB7RP1 antibody used to construct the bispecific constructs described in Example 1, does not bind to murine B7RP1, while the BAFF-binding peptide used in these constructs does bind to both human and murine BAFF. Data not shown. The murine surrogate comprises an antagonistic IgG anti-murine B7RP1 antibody (called “anti-mB7RP1” herein), which was a chimera of mouse immunoglobulin constant regions and rat anti-murine B7RP1 immunoglobulin variable regions. The use of anti-mB7RP1 is described in Hu et al. (2009), J. Immunol. 182:1421, where it is designated 1B7-V2. The murine surrogate has two copies of a BAFF-binding peptide (SEQ ID NO:1) fused via a short linker (five amino acids long) to the C-terminus of the immunoglobulin heavy chain of anti-mB7RP1. The two copies of the BAFF-binding peptide are separated by another linker that is 23 amino acids long. Nucleic acids encoding the heavy chain of the murine surrogate were made using overlap PCR to join nucleic acids encoding the BAFF-binding portion of αBAFF to the downstream end of nucleic acids encoding the heavy chain of 1B7-V2, i.e., anti-mB7RP1.

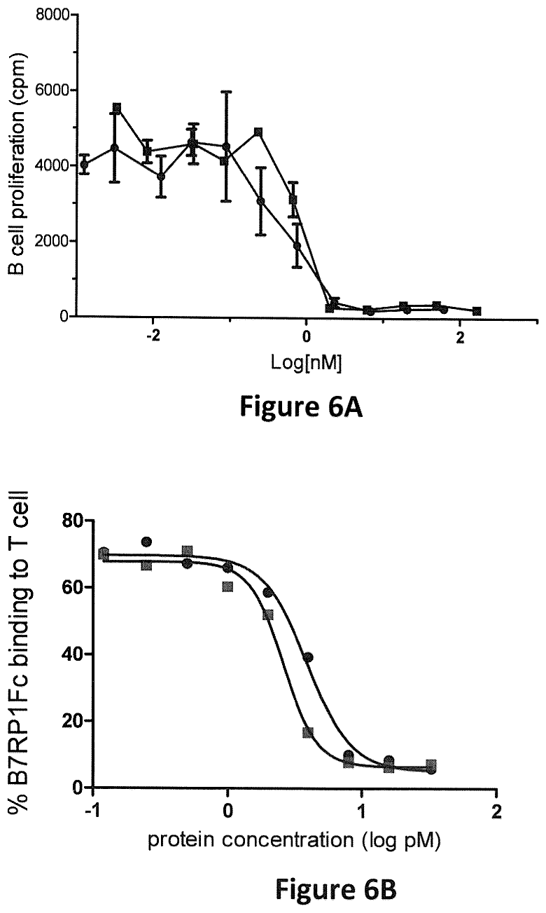

BAFF inhibition by the murine surrogate was evaluated in a BAFF-mediated B cell proliferation assay. Mouse B lymphocytes were isolated from C57BL/6 spleens by negative selection with MACS CD43 (ly-48) Microbeads according to the manufacturers instructions (Miltenyi Biotec, Auburn, CA) or from PBMC using a B cell isolation kit (Miltenyi Biotec, Auburn, CA). Purified B cells were stimulated with 0.1 μg/ml anti-IgM and 200 ng/ml BAFF in the presence of varying concentrations of the murine surrogate or αBAFF. B cell proliferation was measured by 3H-thymidine incorporation at day 4. The IC₅₀'s of the murine surrogate and αBAFF were 0.59 nM and 0.73 nM, respectively. See FIG. 6A. Thus, the murine surrogate effectively inhibited BAFF with potency comparable to that of αBAFF.

To measure inhibition of B7RP1 binding to its receptor by the murine surrogate, mouse spleen cells were first activated to enhance their expression of the B7RP1 receptor by incubating them in microtiter wells coated with an anti-CD3 (5 μg/ml) antibody for 24 hours. The activated spleen cells were washed with phosphate buffered saline (PBS) and then incubated with 5 μg/ml biotinylated muB7RP1: Fc in the presence of varying concentrations of the murine surrogate at 4° C. for 30 minutes. The cells were washed and then stained with allophycocyanin (APC)-conjugated anti-mouse CD3 antibody and streptavidin-phycoerythrin (Streptavidin-PE) for an additional 20 minutes. The B7RP1-Fc binding to T cells was analyzed by flow cytometry. The IC₅₀'s of the murine surrogate and anti-mB7RP1 were 4.01 pM and 2.8 pM, respectively. See FIG. 6B. Hence, the activity of the murine surrogate was similar to that of anti-mB7RP1 in this assay. Thus, the murine surrogate inhibits both BAFF and B7RP1.

The in vivo pharmacodynamic effects of the murine surrogate were evaluated in mice immunized with the sheep red blood cells (SRBC). In brief, BALB/c mice (8 weeks old) received a primary immunization on day 0 and a booster immunization on day 28 with 2×10⁸ SRBC in 0.2 ml of PBS via intraperitoneal injection. The mice (n=5 for each molecule) were treated twice per week from day 0 to day 33 with one of the following molecules at 5 mg/kg: the murine surrogate; αBAFF; anti-mB7RP1; or murine IgG1. Mice treated with SBRC, but not receiving another treatment, served as positive controls. The mice were sacrificed on day 34, and serum and spleens were collected.

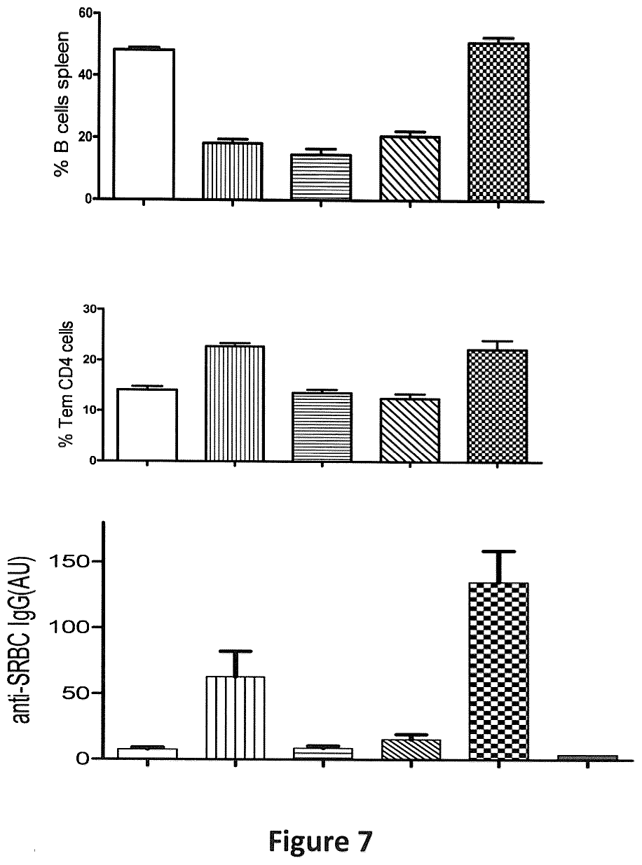

To measure the proportion of B cells and memory T cells in the spleen, spleen cells were harvested by grinding the spleen tissue through a cell strainer. The spleen cells were preincubated with unlabelled anti-CD16/32 to block the nonspecific binding of antibodies to Fc gamma receptors (FcγR). The proportion of B cells was determined by staining with PE-labeled anti-B220 (which is expressed on B cells). The proportion of memory T cells cells (CD44^{hi}CD62L^{lo}CD4 T cells) was determined by staining with FITC-conjugated anti-CD44, PE-conjugated anti-CD62L, APC-conjugated anti-CD4 and PerCP-conjugated anti-CD3. All staining antibodies were purchased from BD Bioscience (San Diego, CA). For both B and T cell determinations, flow cytometry was performed with a FACSCALIBUR™ (BD Bioscience, San Jose, CA) flow cytometer, and the data was analyzed using FLOWJO® (TreeStar Inc., Ashland, OR) software for analysis of flow cytometry data. Results are shown in FIG. 7.

To measure levels of anti-SBRC antibodies in serum, microtiter plates coated with 10 μg/ml soluble SRBC antigen were incubated for two hours at room temperature with diluted serum from treated mice. Bound SRBC-specific Ig from the serum was detected with HRP-conjugated polyclonal goat anti-mouse IgG and IgM antibodies (Southern Biotech, Birmingham, AL). The substrate reaction was performed using SUREBLUE™ TMB microwell peroxidase substrate (KPL, Gaithersburg, MD) according to the manufacturer's instructions, and the optical density was read using a Spectrum Max microplate reader (Molecular Devices). As a positive control, serial dilutions of a mixture of sera from SRBC-immunized mice without any treatment was added to each plate, and a standard curve was constructed from the readings from these wells. Levels of anti-SBRC antibodies of other samples are reported in FIG. 7 as a percentage of this positive control.

The percentage of spleen cells that are B cells was reduced in mice treated with the murine surrogate as compared to the percentage observed in mice treated with murine IgG1. FIG. 7 (top panel). A similar reduction was observed in mice treated with αBAFF or αBAFF plus anti-mB7RP1, but not in mice treated with anti-mB7RP1 alone. FIG. 7 (top panel). With regard to memory T cells, mice treated with the murine surrogate, anti-mB7RP1, or anti-mB7RP1 plus αBAFF had reduced proportions of memory T cells compared to that observed in mice treated with muIgG1. FIG. 7 (middle panel). In contrast, treatment with αBAFF did not alter the memory T cell population in spleen compared to that observed with muIgG treatment. FIG. 7 (middle panel). The murine surrogate also showed potent reduction of the anti-SRBC antibody level in serum, similar to that observed upon treatment with anti-mB7RP1 or anti-mB7RP1 plus αBAFF or in mice that had not been injected with SRBC. FIG. 7 (bottom panel). Moderate inhibition of anti-SRBC antibody level, compared to the level observed with mIgG1 treatment, was observed in mice treated with αBAFF alone. FIG. 7 (bottom panel). These data indicate that the murine surrogate had dual inhibitory effects in B cell and T cell compartments in mice in vivo.

The impact of the murine surrogate on disease was evaluated in the NZB/W F₁ lupus model using two different dose amounts for each of the molecules tested. Female NZB/W F₁ mice (4.5 month old, n=20) were treated twice per week by intraperitoneal injection for 18 weeks using each of the following dosing regimes: 5 or 15 mg/kg murine surrogate (MW≅160 KDa); 4.68 or 14 mg/kg anti-mB7RP1 (MW≅150 KDa); 1.88 or 5.6 mg/kg αBAFF (MW≅64 KDa); a combination of αBAFF (1.88 or 5.6 mg/kg) and anti-mB7RP1 (4.68 or 14 mg/kg); murine IgG1 (15 mg/kg; an isotype control); or phosphate buffered saline (PBS) (a negative control). Proteinuria was measured in urine using ALBUSTIX® (Bayer, Elkhart, IN) every two weeks starting at 5 months of age. The incidence of proteinuria was expressed as the percentage of mice with urine protein at a concentration of at least 300 mg/dl in two consecutive measurements. Serum anti-dsDNA IgG level was measured by ELISA. Scoring for kidney disease of all mice was performed by examination of kidney tissue samples for eight different kinds of lesions, that is, glomerular capillary proliferation, mesangial cell hyperplasia, increased mesangial matrix, glomerular tuft adhesion, parietal epithelial hyperplasia, interstitial nephritis, tubular dilation/protein casts, and tubular atrophy/interstitial fibrosis. Each type of lesion was given a severity score from 0 to 5, for a maximum possible score of 32. The scores of each group of mice were averaged. Survival was monitored.

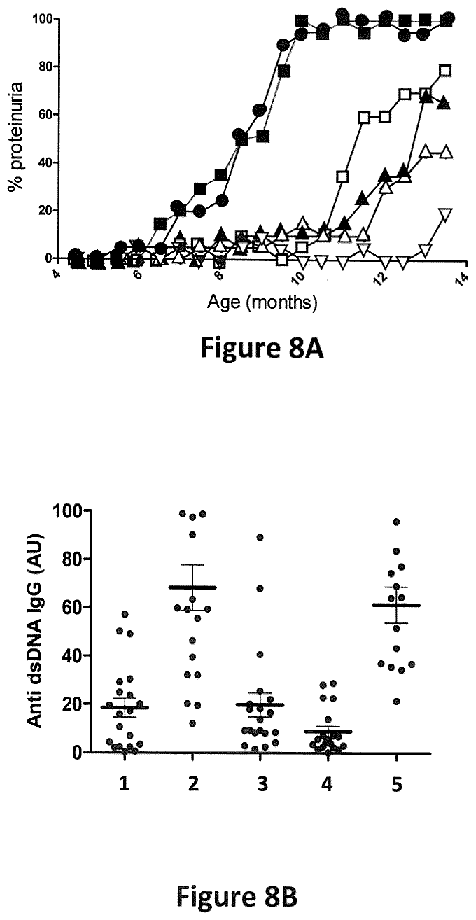

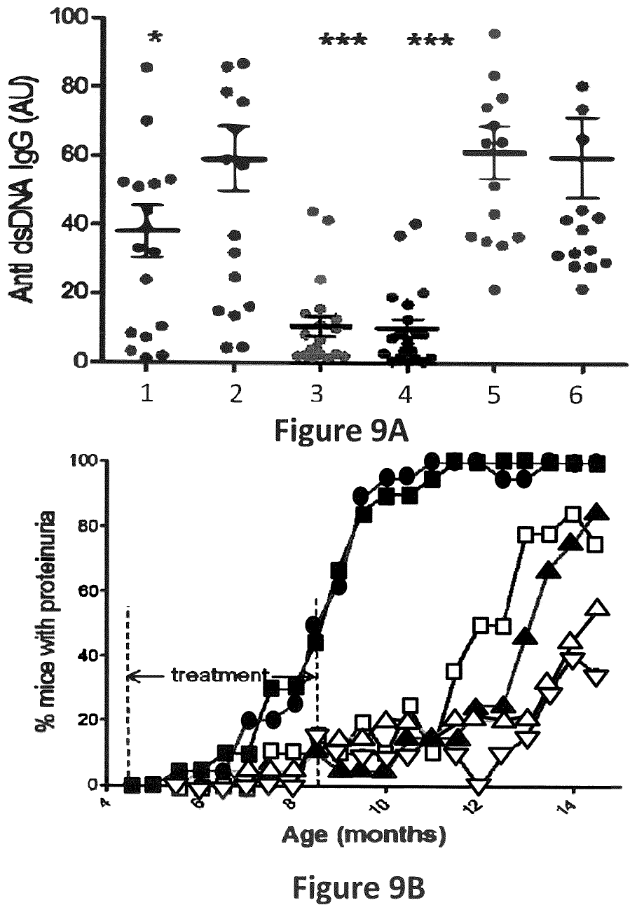

At 12 months of age, none of the mice treated with the murine surrogate at either dose level developed proteinuria. In contrast, 100% of mice treated with murine IgG1 or PBS at both dose levels tested exhibited proteinuria. FIGS. 8A and 9B. About 60% and 35% of mice treated with the lower dose levels of anti-mB7RP1 and αBAFF, respectively, and about 50% and 25% of mice treated with the higher dose levels of anti-mB7RP1 and αBAFF, respectively, developed proteinuria. FIGS. 8A and 9B. In addition, the murine surrogate treatment at both dose levels resulted in a significant reduction in serum levels of anti-dsDNA IgG as compared to the negative control treated with muIgG1. FIGS. 8B and 9A. The bispecific treatment also significantly improved survival compared with the mIgG and PBS control groups. Data not shown. However, no clear difference in survival was observed between the bispecific vs. the single agent treatments at the time of experiment termination.

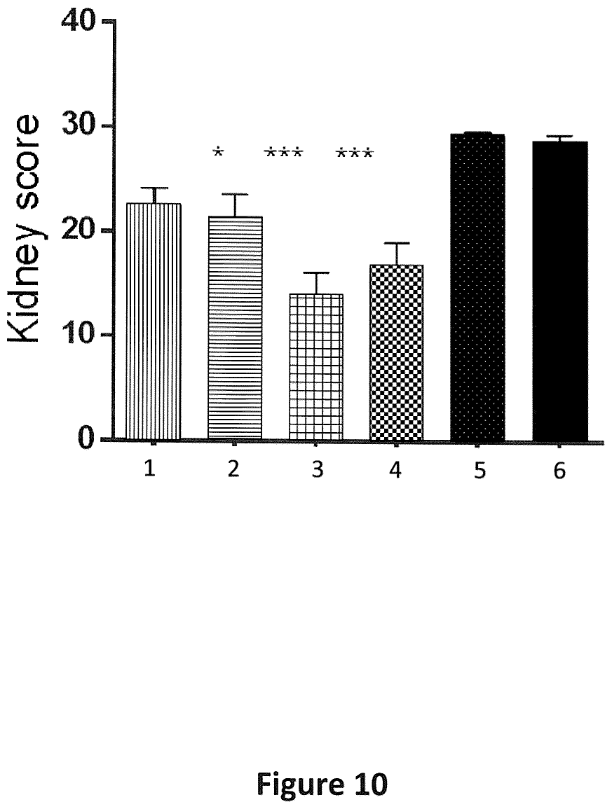

Kidneys from all treated mice, including mice deceased before the end of study, were collected for histology scoring for severity of kidney disease. The groups of mice treated with αBAFF, the combination of αBAFF plus anti-mB7RP1, or the murine surrogate had significantly lower scores for kidney disease as compared to the control group treated with mIgG1. FIG. 10. Groups treated with the surrogate bispecific or the combination also showed a trend towards reduced kidney pathology compared to the single agent treatment groups, a result that correlates well with the proteinuria results described above. Compare FIG. 10 to FIGS. 8A and 9B. In summary, dual inhibition of BAFF and B7RP1 by the murine surrogate or by a combination treatment with αBAFF plus anti-mB7RP1 was more effective than inhibition of only BAFF (αBAFF) or only B7RP1 (anti-mB7RP1) in preventing disease onset and progression in the NZB/W F₁ lupus model.

To determine whether inhibition of both BAFF and B7RP1 could effectively inhibit the symptoms of murine collagen-induced arthritis, the following experiment was done. Male DBA mice were immunized with 100 μg of bovine type II collagen emulsified in 2× Complete Freund's adjuvant (CFA) on day 0 and boosted with bovine type II collagen in Incomplete Freund's Adjuvant (IFA) on day 21. Mice were treated with one of the test substances twice per week during the 41 week course of the study starting on day 0. The percentage of mice in each group exhibiting arthritis symptoms and an average arthritic score for each group was assessed at each time point. Arthritis scores were determined by examining each limb and assigning a score from 0-3 for each limb, with higher scores for more swollen and/or inflamed limbs. So the maximum total arthritis score is 12. A mouse was counted as having arthritis if it had an arthritis score of at least 1 in any limb.

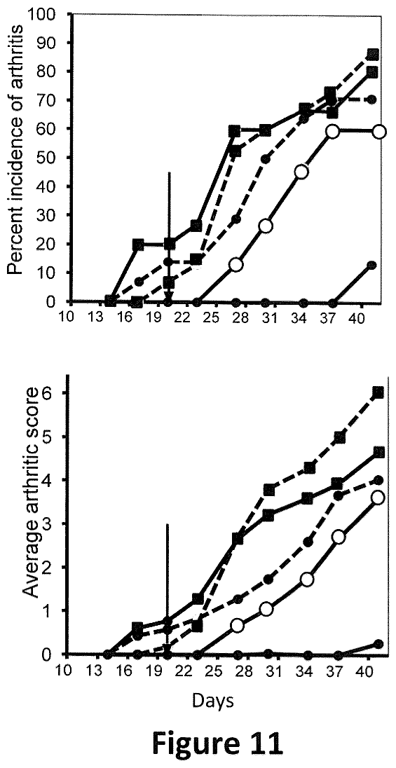

Results are shown in FIG. 11. These data indicate that the combination of αBAFF and anti-mB7RP1 (filled circles connected by solid lines) was much more effective at suppressing arthritis symptoms than either αBAFF (open circles connected by solid lines) or anti-mB7RP1 (filled circles connected by dashed lines) alone. The negative control groups treated with mIgG (filled squares connected by solid lines) or PBS (filled squares connected by dashed lines) had the highest percent incidence of arthritis and highest arthritic scores. These results suggest that inhibiting both BAFF and B7RP1, as opposed to inhibiting only one of these pathways, could be an effective treatment of an autoimmune and/or inflammatory arthritic condition such as rheumatoid arthritis.

1-46. (canceled)

47. A method of treatment comprising treating a patient with a therapeutically effective amount of bispecific protein that can bind B7RP1 and BAFF,
wherein the patient has an autoimmune or inflammatory condition mediated by both T cells and B cells; and
wherein the bispecific protein comprises:
(a) an anti-B7RP1 antibody that comprises a light chain complementarity determining region 1 (CDR1) comprising the amino acid sequence of SEQ ID NO:8, a light chain complementarity determining region 2 (CDR2) comprising the amino acid sequence of SEQ ID NO:9, a light chain complementarity determining region 3 (CDR3) comprising the amino acid sequence of SEQ ID NO:10, a heavy chain CDR1 comprising the amino acid sequence of SEQ ID NO:11, a heavy chain CDR2 comprising the amino acid sequence of SEQ ID NO:12, and a heavy chain CDR3 comprising the amino acid sequence of SEQ ID NO: 13; and
(b) at least one BAFF-binding peptide that is 10 to 40 amino acids long.

48. The method of claim 47, wherein the condition is selected from the group consisting of ANCA-positive vasculitis, rheumatoid arthritis (RA), Crohn's disease, ulcerative colitis, celiac disease, pemphigus, pemphigoid, systemic lupus erythematosus (SLE), lupus nephritis, subacute cutaneous lupus erythematosus (SCLE), multiple sclerosis, chronic inflammatory demyelinating polyneuropathy (CIDP), myasthenia gravis, Goodpasture's syndrome, glomerulonephritis, autoimmune hemolytic anemia (AIHA), idiopathic thrombocytopenia purpura (ITP), chronic active hepatitis, primary biliary cirrhosis, Sjogren's syndrome, systemic sclerosis, Hashimoto's thyroiditis, Graves' disease, Addison's disease, and multiple endocrine neoplasia (MEN).

49. The method of claim 48, wherein the condition is SLE or lupus nephritis.

50. The method of claim 47, wherein the anti-B7RP1 antibody comprises two immunoglobulin heavy chains and two immunoglobulin light chains.

51. The method of claim 47, wherein the anti-B7RP1 antibody is a human or humanized antibody.

52. The method of claim 47, wherein anti-B7RP1 antibody is an IgG antibody.

53. The method of claim 52, wherein the IgG antibody is an IgG1, IgG2, or IgG3 antibody.

54. The method of claim 47, wherein at least one BAFF-binding peptide has the amino acid sequence of SEQ ID NO: 1.

55. The method of claim 50, wherein the anti-B7RP1 antibody's immunoglobulin heavy chains are each linked to at least one BAFF-binding peptide that is 10 to 40 amino acids long.

56. The method of claim 55, wherein each of the immunoglobulin heavy chains are linked to two BAFF-binding peptides that are each 10 to 40 amino acids long.

57. The method of claim 56, wherein the BAFF-binding peptides are attached to the C-terminus of the immunoglobulin heavy chain.

58. The method of claim 57, wherein the polypeptides comprising an immunoglobulin heavy chain and two BAFF-binding peptides have an amino acid sequence of the formula A-L1-P-L2-P;
wherein A is an immunoglobulin heavy chain, L1 is a first linker that is absent or is 3 to 40 amino acids long, P is a BAFF-binding peptide that is 10 to 40 amino acids long, and L2 is a second linker that is absent or is 5 to 50 amino acids long.

59. The method of claim 58, wherein each of the BAFF-binding peptides has the amino acid sequence of SEQ ID NO: 1.

60. The method of claim 58, wherein L1 has the amino acid sequence of SEQ ID NO: 4, SEQ ID NO: 37, SEQ ID NO: 38, SEQ ID NO: 39, or SEQ ID NO: 40.

61. The method of claim 58, wherein L2 has the amino acid sequence of SEQ ID NO: 5, SEQ ID NO: 6, SEQ ID NO: 7, or SEQ ID NO: 24.

62. The method of claim 47, wherein the anti-B7RP1 antibody comprises a VL region comprising the amino acid sequence of SEQ ID NO: 14.

63. The method of claim 47, wherein the anti-B7RP1 antibody comprises a VH region comprising the amino acid sequence of SEQ ID NO:15.

64. The method of claim 47, wherein the anti-B7RP1 antibody comprises a heavy chain comprising the amino acid sequence of SEQ ID NO: 17 or SEQ ID NO: 18; and wherein the anti-B7RP1 antibody comprises a light chain comprising the amino acid sequence of SEQ ID NO: 19.

65. The method of claim 47, wherein the bispecific protein binds to human BAFF with an affinity (K_{D}) of approximately 30 pM, as measured by Kinetic Exclusion Assay.

66. The method of claim 47, wherein the bispecific protein binds to human B7RP1 with an affinity (K_{D}) of approximately 40 pM, as measured by Kinetic Exclusion Assay.
